(19) United States
(12) Patent Application Publication
(10) Pub. No.: US 2025/0137056 A1
(43) Pub. Date: May 1, 2025

(54) ASSAY AND RELATED METHODS, KITS AND DEVICES

(71) Applicant: Bio Preventive Medicine Corp., Zhubei City (TW)

(72) Inventors: Tzu-Ling Tseng, Zhubei City (TW); Chi-Hsuan Huang, Hsinchu (TW); Cherng-Shyang Chang, Hsinchu (TW); Yan-Shen Shan, Tainan (TW)

(21) Appl. No.: 18/385,999

(22) Filed: Nov. 1, 2023

(51) Int. Cl. C12Q 1/6886; C12Q 1/6806; C12Q 1/6851

(52) U.S. Cl. CPC C12Q 1/6886; G16B 25/10; C12Q 2600/158; C12Q 2600/166; C12Q 2600/178; G01N 2800/50; G01N 2800/54

(57) ABSTRACT

The present disclosure is related to a method of assessing a probability of an occurrence of a cancer, in which the method may comprise obtaining respective cancer-associated polynucleotide levels based on a plurality of different cancer-associated polynucleotide molecules and respective reference polynucleotide levels based on a plurality of different reference polynucleotide molecules, adjusting the respective cancer-associated polynucleotide levels based on the respective reference polynucleotide levels, assigning coefficients to the adjusted cancer-associated polynucleotide levels, respectively, and assessing the probability of the occurrence of the cancer, based on the adjusted cancer-associated polynucleotide levels and the assigned coefficients.

### BACKGROUND

Cancer is a complex and widespread disease that affects millions of people around the world. Cancer can develop in various parts of the body, including the lungs, breast, prostate, colon, skin, and many others. The development of cancer is a multistep process. It usually begins with the transformation of a normal cell into a cancerous cell, a process known as oncogenesis. This transformation can occur due to mutations or alterations in the DNA of the cell. These genetic changes can disrupt the normal regulation of cell growth, leading to uncontrolled proliferation. Prevention and early detection play crucial roles in the fight against cancer. Detecting the disease at its earlier stage can allow more treatment options.

### SUMMARY

This Summary is provided to introduce a selection of concepts in simplified form that are further described below in the Detailed Description. This Summary is not intended to identify key aspects or essential aspects of the claimed subject matter.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with any accompanying Figures.

The present disclosure is related to a method of assessing a probability of an occurrence of a cancer, in which the method comprises obtaining respective cancer-associated polynucleotide levels based on a plurality of different cancer-associated polynucleotide molecules and respective reference polynucleotide levels based on a plurality of different reference polynucleotide molecules, wherein the plurality of different cancer-associated polynucleotide molecules and the plurality of reference polynucleotide molecules may be generated based on a plurality of agents added to a solution containing a plurality of different cancer-associated miRNAs present in a bodily fluid sample and a plurality of different reference miRNAs, wherein the plurality of agents may be to interact with the plurality of different cancer-associated miRNAs and the plurality of different reference miRNAs; adjusting the respective cancer-associated polynucleotide levels based on the respective reference polynucleotide levels; assigning coefficients to the adjusted cancer-associated polynucleotide levels, respectively; and assessing the probability of the occurrence of the cancer, based on the adjusted cancer-associated polynucleotide levels and the assigned coefficients.

The present disclosure is related to a method of preparing adjusted cancer-associated polynucleotide levels useful for assessing a probability of an occurrence of a cancer, in which the method comprises obtaining respective cancer-associated polynucleotide levels based on a plurality of different cancer-associated polynucleotide molecules and respective reference polynucleotide levels based on a plurality of different reference polynucleotide molecules, wherein the plurality of different cancer-associated polynucleotide molecules and the plurality of reference polynucleotide molecules may be generated based on a plurality of agents added to a solution containing a plurality of different cancer-associated miRNAs present in a bodily fluid sample and a plurality of different reference miRNAs, wherein the plurality of agents may be to interact with the plurality of different cancer-associated miRNAs and the plurality of different reference miRNAs; adjusting the respective cancer-associated polynucleotide levels based on the respective reference polynucleotide levels; assigning coefficients to the adjusted cancer-associated polynucleotide levels, respectively; and assessing the probability of the occurrence of the cancer, based on the adjusted cancer-associated polynucleotide levels and the assigned coefficients.

In some embodiments, the respective cancer-associated polynucleotide levels may indicate miRNA levels of the plurality of different cancer-associated miRNAs and respective reference polynucleotide levels may indicate reference miRNA levels of the plurality of different reference miRNAs.

In some embodiments, the method may further comprise adding the plurality of agents to the solution containing the plurality of different cancer-associated miRNAs and the plurality of different reference miRNAs.

In some embodiments, the plurality of different reference miRNAs may include an exogenous miRNA added to the bodily fluid sample. In some embodiments, the plurality of different reference miRNAs may include an endogenous miRNA. In some embodiments, the plurality of different reference miRNAs may include a plurality of endogenous miRNAs.

In some embodiments, the plurality of different cancer-associated polynucleotide molecules may include a plurality of different copy DNAs (cDNAs) amplified from a plurality of different cancer-associated miRNAs present in a bodily fluid sample.

In some embodiments, the plurality of different reference polynucleotide molecules may include a plurality of different reference copy DNAs (cDNAs) amplified from a plurality of different reference miRNAs.

In some embodiments, the cancer may be a gastrointestinal (GI) cancer.

In some embodiments, the plurality of different cancer-associated miRNAs may include hsa-miR-191-5p, hsa-miR-21-5p, hsa-miR-19a-3p, hsa-miR-145-5p, hsa-miR-23a-3p, or any combination thereof. In some embodiments, the plurality of reference miRNAs may include cel-miR-39-3p, hsa-miR-16-5p, hsa-miR-484, or any combination thereof. In some embodiments, the exogenous miRNA may include cel-miR-39-3p.

In some embodiments, the respective cancer-associated polynucleotide levels may be adjusted based on the respective reference polynucleotide levels by subtracting a reference polynucleotide level among the respective reference polynucleotide levels from a cancer-associated polynucleotide level among the respective cancer-associated polynucleotide levels, to adjust the cancer-associated polynucleotide level.

In some embodiments, the plurality of different cancer-associated miRNAs or the plurality of different reference miRNAs may include an exosomal RNA, a circulating miRNA, or a combination thereof.

In some embodiments, the probability of the occurrence of the cancer may be used in a prognostic analysis, a diagnostic analysis, or a combination thereof.

In some embodiments, the step of assessing the probability of the occurrence of the cancer may be based on Equation (1):

$$y = a 0 + a 1 * BB + a 2 * EE + a 3 * FF + a 4 * GG + a 5 * HH \qquad Equation ( 1 )$$

wherein
y is a first probabilistic value to be based on to assess the probability of the occurrence of the cancer,
BB, EE, FF, GG, and HH are values corresponding to the adjusted cancer-associated polynucleotide levels, and
a0, a1, a2, a3, a4, and a5 are the assigned coefficients.

In some embodiments, BB corresponds to hsa-miR-191-5p, EE corresponds to the hsa-miR-21-5p, FF corresponds to the hsa-miR-19a-3p, GG corresponds to the hsa-miR-145-5p, and HH corresponds to the hsa-miR-23a-3p.

In some embodiments, the step of adjusting the respective cancer-associated polynucleotide levels based on the respective reference polynucleotide levels may further comprise assigning reference coefficients to the respective reference polynucleotide levels; adjusting the respective reference polynucleotide levels with the assigned reference coefficients; and adjusting the respective cancer-associated polynucleotide levels based on the adjusted respective reference polynucleotide levels.

In some embodiments, the step of assessing the probability of the occurrence of the cancer may be based on Equation (3):

$$p = exp ( y ) / ( 1 + exp ( y ) ) \qquad Equation ( 3 )$$

wherein p is a second probabilistic value to be based on to assess the probability of the occurrence of the cancer.

In some embodiments, adjusting the respective reference polynucleotide levels may be based on Equation (2):

$$α1 * AA + α2 * CC + α3 * DD \qquad Equation ( 2 )$$

wherein AA, CC, and DD are values corresponding to the respective reference polynucleotide levels, and
α1, α2, and α3 are the reference coefficients.

In some embodiments, AA corresponds to cel-miR-39-3p, CC corresponds to hsa-miR-16-5p, and DD corresponds to hsa-miR-484.

In some embodiments, the step of assessing the probability of the occurrence of the cancer can be based on Equation (4):

$$y = a 0 + a 1 * [ BB - ( α1 * AA + α2 * CC + α3 * DD ) ] + a 2 * [ E E - ( α1 * AA + α2 * CC + α3 * DD ) ] + a 3 * [ F F - ( α1 * AA + α2 * CC + α3 * DD ) ] + a {4}^{*} [ G G - ( α1 * AA + α2 * CC + α3 * DD ) ] + a 5 * [ H H - ( α1 * AA + α2 * CC + α3 * DD ) ] \qquad Equation ( 4 )$$

wherein
y is a first probabilistic value to be based on to assess the probability of the occurrence of the cancer,
BB, EE, FF, GG, and HH are values corresponding to the respective cancer-associated polynucleotide levels,
a0, a1, a2, a3, a4, and a5 are the assigned coefficients,
AA, CC, and DD are values corresponding to the respective reference polynucleotide levels, and
α1, α2, and α3 are the reference coefficients.

In some embodiments, AA corresponds to cel-miR-39-3p, CC can correspond to hsa-miR-16-5p, and DD can correspond to hsa-miR-484.

In some embodiments, the step of assessing the probability of the occurrence of the cancer may be based on Equation (3):

$$p = exp ( y ) / ( 1 + exp ( y ) ) \qquad Equation ( 3 )$$

wherein p is a second probabilistic value to be based on to assess the probability of the occurrence of the cancer.

In some embodiments, the method may further comprise, in response to the second probabilistic value exceeding a threshold value, determining that a patient from whom the bodily fluid was derived from has an elevated risk of cancer.

In some embodiments, a0 value may be from about 2.66 to about 7.03; a1 value may be 0; a2 value may be from about −1.47 to about 0.03; a3 value may be from about 2.17 to about 4.11; a4 value may be from about −1.57 to about −0.58; a5 value may be from about 0.32 to about 1.46; α1 value may be 0; α2 value may be from about −0.43 to about 0.34; and α3 value may be from about 0.59 to about 1.58.

In some embodiments, a0 value may be from about 2.66 to about 7.04; a1 value may be from about −0.44 to about 0.43; a2 value may be from about −1.47 to about 0.04; a3 value may be from about 2.14 to about 4.15; a4 value may be from about −1.58 to about −0.57; a5 value may be from about 0.26 to about 1.53; α1 value may be 0; α2 value may be from about −0.48 to about 0.38; and α3 value may be from about 0.59 to about 1.6.

In some embodiments, a0 value may be from about −0.41 to about 2.03; a1 value may be from about 0.03 to about 0.87; a2 value may be 0; a3 value may be from about 1.73 to about 3.24; a4 value may be from about −1.53 to about −0.6; a5 value may be 0; α1 value may be from about −0.91 to about −0.13; α2 value may be 0; and α3 value may be from about 0.8 to about 1.79.

In some embodiments, a0 value may be from about 0.39 to about 2.64; a1 value may be from about 0.01 to about 0.83; a2 value may be 0; a3 value may be from about 1.67 to about 3.12; a4 value may be from about −1.52 to about −0.59; a5 value may be 0; α1 value may be from about −1.01 to about −0.15; α2 value may be from about −0.62 to about 0.41; and α3 value may be from about 0.84 to about 2.02.

In some embodiments, a0 value may be from about 4.09 to about 8.49; a1 value may be 0; a2 value may be from about −0.9 to about 0.2; a3 value may be from about 1.47 to about 2.89; a4 value may be from about −1.35 to about −0.43; a5 value may be 0; α1 value may be from about −1.83 to about −0.24; α2 value may be from about −1.41 to about 0.43; and α3 value may be from about 1.22 to about 3.16.

In some embodiments, a0 value may be from about 1.96 to about 3.85; a1 value may be 0; a2 value may be 0; a3 value may be from about 1.59 to about 2.99; a4 value may be from about −1.32 to about −0.51; a5 value may be about 0; α1 value may be from about −1.09 to about −0.09; α2 value may be 0; and α3 value may be from about 0.89 to about 1.85.

In some embodiments, a0 value may be from about 2.91 to about 6.5; a1 value may be 0; a2 value may be from about −0.87 to about 0.21; a3 value may be from about 1.61 to about 3.12; a4 value may be from about −1.35 to about −0.43; a5 value may be 0; α1 value may be from about −1.36 to about −0.13; α2 value may be 0; and α3 value may be from about 0.94 to about 2.15.

In some embodiments, a0 value may be from about 3.07 to about 5.54; a1 value may be 0; a2 value may be about 0; a3 value may be from about 1.45 to about 2.74; a4 value may be from about −1.32 to about −0.51; a5 value may be 0; α1 value may be from about −1.39 to about −0.16; α2 value may be from about −1.1 to about 0.36; and α3 value may be from about 1.07 to about 2.57.

In some embodiments, a0 value may be from about 1.31 to about 3.06; a1 value may be 0; a2 value may be 0; a3 value may be from about 2 to about 3.78; a4 value may be from about −1.58 to about −0.6; a5 value may be from about 0.25 to about 1.16; α1 value may be 0; α2 value may be from about −0.39 to about 0.29; and α3 value may be from about 0.6 to about 1.46.

In some embodiments, a0 value may be from about 1.06 to about 3.31; a1 value may be from about −0.42 to about 0.43; a2 value may be 0; a3 value may be from about 1.97 to about 3.82; a4 value may be from about −1.58 to about −0.6; a5 value may be from about 0.2 to about 1.2; a1 value may be 0; a2 value may be from about −0.43 to about 0.32; and a3 value may be from about 0.59 to about 1.46.

In some embodiments, a0 value may be from about 4.47 to about 8.1; a1 value may be from about −0.05 to about 0.69; a2 value may be 0; a3 value may be from about 2 to about 3.82; a4 value may be from about −1.39 to about −0.52; a5 value may be 0; α1 value may be 0; α2 value may be from about −0.34 to about 0.44; and α3 value may be from about 0.57 to about 1.48.

In some embodiments, a0 value may be from about 5.23 to about 10.96; a1 value may be from about −0.04 to about 0.73; a2 value may be from about −0.92 to about 0.23; a3 value may be from about 2.07 to about 4; a4 value may be from about −1.4 to about −0.47; a5 value may be 0; α1 value may be 0; α2 value may be from about −0.36 to about 0.5; and α3 value may be from about 0.56 to about 1.56.

In some embodiments, a0 value may be from about 6.24 to about 12.73; a1 value may be 0; a2 value may be from about −0.74 to about 0.31; a3 value may be from about 1.83 to about 3.47; a4 value may be from about −1.24 to about −0.39; a5 value may be 0; α1 value may be 0; α2 value may be from about −0.58 to about 0.44; and α3 value may be from about 0.64 to about 1.8.

In some embodiments, a0 value may be from about 5.86 to about 10.61; a1 value may be 0; a2 value may be 0; a3 value may be from about 1.81 to about 3.38; a4 value may be from about −1.22 to about −0.45; a5 value may be 0; α1 value may be 0; α2 value may be from about −0.54 to about 0.41; and α3 value may be from about 0.65 to about 1.68.

In some embodiments, a0 value may be from about −8.04 to about −3.13; a1 value may be 0; a2 value may be from about −2.27 to about −0.62; a3 value may be from about 1.25 to about 2.59; a4 value may be 0; a5 value may be from about 0.26 to about 1.49; α1 value may be from about −1.53 to about −0.27; α2 value may be from about −1.04 to about 0.12; and α3 value may be from about 1.02 to about 2.53.

In some embodiments, a0 value may be from about −8.2 to about −2.68; a1 value may be from about −0.46 to about 0.33; a2 value may be from about −2.26 to about −0.61; a3 value may be from about 1.23 to about 2.57; a4 value may be 0; a5 value may be from about 0.26 to about 1.56; α1 value may be from about −1.57 to about −0.26; α2 value may be from about −1.15 to about 0.14; and α3 value may be from about 1.04 to about 2.61.

In some embodiments, a0 value may be from about −9.01 to about −3.7; a1 value may be 0; a2 value may be from about −2.21 to about −0.6; a3 value may be from about 1.42 to about 2.88; a4 value may be 0; a5 value may be from about 0.21 to about 1.38; α1 value may be from about −1.13 to about −0.14; α2 value may be 0; and α3 value may be from about 0.75 to about 1.76.

In some embodiments, a0 value may be from about −9.55 to about −3.35; a1 value may be from about −0.33 to about 0.5; a2 value may be from about −2.22 to about −0.6; a3 value may be from about 1.43 to about 2.88; a4 value may be 0; a5 value may be from about 0.13 to about 1.36; α1 value may be from about −1.14 to about −0.14; α2 value may be 0; and α3 value may be from about 0.72 to about 1.81.

In some embodiments, a0 value may be from about −1.89 to about 1.02; a1 value may be from about −0.51 to about 0.22; a2 value may be from about −1.7 to about −0.21; a3 value may be from about 1.56 to about 3.11; a4 value may be 0; a5 value may be from about 0.1 to about 1.25; α1 value may be 0; α2 value may be from about −0.61 to about 0.24; and α3 value may be from about 0.6 to about 1.64.

In some embodiments, a0 value may be from about −1.95 to about 0.76; a1 value may be 0; a2 value may be from about −1.71 to about −0.21; a3 value may be from about 1.64 to about 3.19; a4 value may be 0; a5 value may be from about 0.06 to about 1.1; α1 value may be 0; α2 value may be from about −0.47 to about 0.24; and α3 value may be from about 0.58 to about 1.56.

The present disclosure is related to a method of assessing a probability of an occurrence of a cancer, in which the method comprises adding an exogenous reference miRNA to a bodily fluid sample including a plurality of different cancer-associated miRNAs present in the bodily fluid sample; analyzing the bodily fluid sample, to determine miRNA levels of the plurality of different cancer-associated miRNAs and at least one reference miRNA level of at least one reference miRNA including the exogenous reference miRNA; adjusting at least one miRNA level among the miRNA levels based on the at least one reference miRNA level; assigning coefficients to the miRNA levels including the at least one adjusted miRNA level; and assessing the probability of the occurrence of the cancer, based on the miRNA levels and the assigned coefficients.

The present disclosure is related to a method of preparing at least one adjusted miRNA level useful for assessing a probability of an occurrence of a cancer, in which the method comprises adding an exogenous reference miRNA to a bodily fluid sample including a plurality of different cancer-associated miRNAs present in the bodily fluid sample; analyzing the bodily fluid sample, to determine miRNA levels of the plurality of different cancer-associated miRNAs and at least one reference miRNA level of at least one reference miRNA including the exogenous reference miRNA; adjusting at least one miRNA level among the miRNA levels based on the at least one reference miRNA level; assigning coefficients to the miRNA levels including the at least one adjusted miRNA level; and assessing the probability of the occurrence of the cancer, based on the miRNA levels and the assigned coefficients.

In some embodiments, the at least one reference miRNA may include a plurality of different reference miRNAs, and the method may further comprise analyzing the bodily fluid sample including the exogenous miRNA, to determine reference miRNA levels of the plurality of different reference miRNAs; adjusting corresponding miRNA levels among the miRNA levels based on the reference miRNA levels; assigning coefficients to the miRNA levels including the adjusted corresponding miRNA levels; and assessing the probability of the occurrence of the cancer, based on the miRNA levels and the assigned coefficients.

In some embodiments, the plurality of different reference miRNAs may include an endogenous miRNA.

In some embodiments, the plurality of different reference miRNAs may include a plurality of endogenous miRNAs.

In some embodiments, the miRNA levels of the plurality of different cancer-associated miRNAs may be determined based on a plurality of different copy DNAs (cDNAs) amplified from the plurality of different cancer-associated miRNAs. In some embodiments, the reference miRNA level of the reference miRNA a reference copy DNA (cDNA) amplified from the reference miRNA.

In some embodiments, the cancer may be a gastrointestinal (GI) cancer.

In some embodiments, the plurality of different cancer-associated miRNAs may include hsa-miR-191-5p, hsa-miR-21-5p, hsa-miR-19a-3p, hsa-miR-145-5p, hsa-miR-23a-3p, or any combination thereof. In some embodiments, the at least one reference miRNA may include cel-miR-39-3p, hsa-miR-16-5p, hsa-miR-484, or any combination thereof.

In some embodiments, the respective cancer-associated miRNA levels may be adjusted based on the respective reference miRNA levels by subtracting a reference miRNA level among the respective reference miRNA levels from a cancer-associated miRNA level among the respective cancer-associated miRNA levels, to adjust the cancer-associated miRNA level.

In some embodiments, the plurality of different cancer-associated miRNAs or the plurality of different reference miRNAs may include an exosomal RNA, a circulating miRNA, or a combination thereof.

In some embodiments, the probability of the occurrence of the cancer may be used in a prognostic analysis, a diagnostic analysis, or a combination thereof.

In some embodiments, the step of assessing the probability of the occurrence of the cancer may be based on Equation (1):

$$y = a 0 + a 1 * BB + a 2 * EE + a 3 * FF + a 4 * GG + a 5 * HH \qquad Equation ( 1 )$$

wherein
   y is a first probabilistic value to be based on to assess the probability of the occurrence of the cancer,
   BB, EE, FF, GG, and HH are values corresponding to the adjusted cancer-associated miRNA levels,
   and a0, a1, a2, a3, a4, and a5 are the assigned coefficients.

In some embodiments, BB corresponds to hsa-miR-191-5p, EE corresponds to the hsa-miR-21-5p, FF corresponds to the hsa-miR-19a-3p, GG corresponds to the hsa-miR-145-5p, and HH corresponds to the hsa-miR-23a-3p.

In some embodiments, the step of adjusting the respective cancer-associated miRNA levels based on the respective reference miRNA levels may further comprises assigning reference coefficients to the respective reference miRNA levels; adjusting the respective reference miRNA levels with the assigned reference coefficients; and adjusting the respective cancer-associated miRNA levels based on the adjusted respective reference miRNA levels.

In some embodiments, the step of assessing the probability of the occurrence of the cancer may be based on Equation (3):

$$p = exp ( y ) / ( 1 + exp ( y ) ) \qquad Equation ( 3 )$$

wherein p is a second probabilistic value to be based on to assess the probability of the occurrence of the cancer.

In some embodiments, adjusting the respective reference polynucleotide levels may be based on Equation (2):

$$α1 * AA + α2 * CC + α3 * DD \qquad Equation ( 2 )$$

wherein AA, CC, and DD are values corresponding to the respective reference polynucleotide levels, and
α1, α2, and α3 are the reference coefficients.

In some embodiments, AA corresponds to cel-miR-39-3p, CC corresponds to hsa-miR-16-5p, and DD corresponds to hsa-miR-484.

In some embodiments, the step of assessing the probability of the occurrence of the cancer can be based on Equation (4):

$$y = a 0 + a 1 * [ BB - ( α1 * AA + α2 * CC + α3 * DD ) ] + a 2 * [ E E - ( α1 * AA + α2 * CC + α3 * DD ) ] + a 3 * [ F F - ( α1 * AA + α2 * CC + α3 * DD ) ] + a {4}^{*} [ G G - ( α1 * AA + α2 * CC + α3 * DD ) ] + a 5 * [ H H - ( α1 * AA + α2 * CC + α3 * DD ) ] \qquad Equation ( 4 )$$

wherein
y is a first probabilistic value to be based on to assess the probability of the occurrence of the cancer,
BB, EE, FF, GG, and HH are values corresponding to the respective cancer-associated polynucleotide levels,
a0, a1, a2, a3, a4, and a5 are the assigned coefficients,
AA, CC, and DD are values corresponding to the respective reference polynucleotide levels, and
α1, α2, and α3 are the reference coefficients.

In some embodiments, AA corresponds to cel-miR-39-3p, CC can correspond to hsa-miR-16-5p, and DD can correspond to hsa-miR-484.

In some embodiments, the step of assessing the probability of the occurrence of the cancer may be based on Equation (3):

$$p = exp ( y ) / ( 1 + exp ( y ) ) \qquad Equation ( 3 )$$

wherein p is a second probabilistic value to be based on to assess the probability of the occurrence of the cancer.

In some embodiments, the method may further comprise, in response to the second probabilistic value exceeding a threshold value, determining that a patient from whom the bodily fluid was derived from has an elevated risk of cancer.

In some embodiments, a0 value may be from about 2.66 to about 7.03; a1 value may be 0; a2 value may be from about −1.47 to about 0.03; a3 value may be from about 2.17 to about 4.11; a4 value may be from about −1.57 to about −0.58; a5 value may be from about 0.32 to about 1.46; α1 value may be 0; α2 value may be from about −0.43 to about 0.34; and α3 value may be from about 0.59 to about 1.58.

In some embodiments, a0 value may be from about 2.66 to about 7.04; a1 value may be from about −0.44 to about 0.43; a2 value may be from about −1.47 to about 0.04; a3 value may be from about 2.14 to about 4.15; a4 value may be from about −1.58 to about −0.57; a5 value may be from about 0.26 to about 1.53; α1 value may be 0; α2 value may be from about −0.48 to about 0.38; and α3 value may be from about 0.59 to about 1.6.

In some embodiments, a0 value may be from about −0.41 to about 2.03; a1 value may be from about 0.03 to about 0.87; a2 value may be 0; a3 value may be from about 1.73 to about 3.24; a4 value may be from about −1.53 to about −0.6; a5 value may be 0; α1 value may be from about −0.91 to about −0.13; α2 value may be 0; and α3 value may be from about 0.8 to about 1.79.

In some embodiments, a0 value may be from about 0.39 to about 2.64; a1 value may be from about 0.01 to about 0.83; a2 value may be 0; a3 value may be from about 1.67 to about 3.12; a4 value may be from about −1.52 to about −0.59; a5 value may be 0; α1 value may be from about −1.01 to about −0.15; α2 value may be from about −0.62 to about 0.41; and α3 value may be from about 0.84 to about 2.02.

In some embodiments, a0 value may be from about 4.09 to about 8.49; a1 value may be 0; a2 value may be from about −0.9 to about 0.2; a3 value may be from about 1.47 to about 2.89; a4 value may be from about −1.35 to about −0.43; a5 value may be 0; α1 value may be from about −1.83 to about −0.24; α2 value may be from about −1.41 to about 0.43; and α3 value may be from about 1.22 to about 3.16.

In some embodiments, a0 value may be from about 1.96 to about 3.85; a1 value may be 0; a2 value may be 0; a3 value may be from about 1.59 to about 2.99; a4 value may be from about −1.32 to about −0.51; a5 value may be about 0; α1 value may be from about −1.09 to about −0.09; α2 value may be 0; and α3 value may be from about 0.89 to about 1.85.

In some embodiments, a0 value may be from about 2.91 to about 6.5; a1 value may be 0; a2 value may be from about −0.87 to about 0.21; a3 value may be from about 1.61 to about 3.12; a4 value may be from about −1.35 to about −0.43; a5 value may be 0; α1 value may be from about −1.36 to about −0.13; α2 value may be 0; and α3 value may be from about 0.94 to about 2.15.

In some embodiments, a0 value may be from about 3.07 to about 5.54; a1 value may be 0; a2 value may be about 0; a3 value may be from about 1.45 to about 2.74; a4 value may be from about −1.32 to about −0.51; a5 value may be 0; α1 value may be from about −1.39 to about −0.16; α2 value may be from about −1.1 to about 0.36; and α3 value may be from about 1.07 to about 2.57.

In some embodiments, a0 value may be from about 1.31 to about 3.06; a1 value may be 0; a2 value may be 0; a3 value may be from about 2 to about 3.78; a4 value may be from about −1.58 to about −0.6; a5 value may be from about 0.25 to about 1.16; α1 value may be 0; α2 value may be from about −0.39 to about 0.29; and α3 value may be from about 0.6 to about 1.46.

In some embodiments, a0 value may be from about 1.06 to about 3.31; a1 value may be from about −0.42 to about 0.43; a2 value may be 0; a3 value may be from about 1.97 to about 3.82; a4 value may be from about −1.58 to about −0.6; a5 value may be from about 0.2 to about 1.2; α1 value may be 0; α2 value may be from about −0.43 to about 0.32; and α3 value may be from about 0.59 to about 1.46.

In some embodiments, a0 value may be from about 4.47 to about 8.1; a1 value may be from about −0.05 to about 0.69; a2 value may be 0; a3 value may be from about 2 to about 3.82; a4 value may be from about −1.39 to about −0.52; a5 value may be 0; α1 value may be 0; α2 value may be from about −0.34 to about 0.44; and α3 value may be from about 0.57 to about 1.48.

In some embodiments, a0 value may be from about 5.23 to about 10.96; a1 value may be from about −0.04 to about 0.73; a2 value may be from about −0.92 to about 0.23; a3 value may be from about 2.07 to about 4; a4 value may be from about −1.4 to about −0.47; a5 value may be 0; α1 value may be 0; α2 value may be from about −0.36 to about 0.5; and α3 value may be from about 0.56 to about 1.56.

In some embodiments, a0 value may be from about 6.24 to about 12.73; a1 value may be 0; a2 value may be from about −0.74 to about 0.31; a3 value may be from about 1.83 to about 3.47; a4 value may be from about −1.24 to about −0.39; a5 value may be 0; α1 value may be 0; α2 value may be from about −0.58 to about 0.44; and α3 value may be from about 0.64 to about 1.8.

In some embodiments, a0 value may be from about 5.86 to about 10.61; a1 value may be 0; a2 value may be 0; a3 value may be from about 1.81 to about 3.38; a4 value may be from about −1.22 to about −0.45; a5 value may be 0; α1 value may be 0; α2 value may be from about −0.54 to about 0.41; and α3 value may be from about 0.65 to about 1.68.

In some embodiments, a0 value may be from about −8.04 to about −3.13; a1 value may be 0; a2 value may be from about −2.27 to about −0.62; a3 value may be from about 1.25 to about 2.59; a4 value may be 0; a5 value may be from about 0.26 to about 1.49; α1 value may be from about −1.53 to about −0.27; α2 value may be from about −1.04 to about 0.12; and α3 value may be from about 1.02 to about 2.53.

In some embodiments, a0 value may be from about −8.2 to about −2.68; a1 value may be from about −0.46 to about 0.33; a2 value may be from about −2.26 to about −0.61; a3 value may be from about 1.23 to about 2.57; a4 value may be 0; a5 value may be from about 0.26 to about 1.56; α1 value may be from about −1.57 to about −0.26; α2 value may be from about −1.15 to about 0.14; and α3 value may be from about 1.04 to about 2.61.

In some embodiments, a0 value may be from about −9.01 to about −3.7; a1 value may be 0; a2 value may be from about −2.21 to about −0.6; a3 value may be from about 1.42 to about 2.88; a4 value may be 0; a5 value may be from about 0.21 to about 1.38; α1 value may be from about −1.13 to about −0.14; α2 value may be 0; and α3 value may be from about 0.75 to about 1.76.

In some embodiments, a0 value may be from about −9.55 to about −3.35; a1 value may be from about −0.33 to about 0.5; a2 value may be from about −2.22 to about −0.6; a3 value may be from about 1.43 to about 2.88; a4 value may be 0; a5 value may be from about 0.13 to about 1.36; α1 value may be from about −1.14 to about −0.14; α2 value may be 0; and α3 value may be from about 0.72 to about 1.81.

In some embodiments, a0 value may be from about −1.89 to about 1.02; a1 value may be from about −0.51 to about 0.22; a2 value may be from about −1.7 to about −0.21; a3 value may be from about 1.56 to about 3.11; a4 value may be 0; a5 value may be from about 0.1 to about 1.25; α1 value may be 0; α2 value may be from about −0.61 to about 0.24; and α3 value may be from about 0.6 to about 1.64.

In some embodiments, a0 value may be from about −1.95 to about 0.76; a1 value may be 0; a2 value may be from about −1.71 to about −0.21; a3 value may be from about 1.64 to about 3.19; a4 value may be 0; a5 value may be from about 0.06 to about 1.1; α1 value may be 0; α2 value may be from about −0.47 to about 0.24; and α3 value may be from about 0.58 to about 1.56.

### BRIEF DESCRIPTION OF THE FIGURES

FIG. 1 illustrates a process of analyzing miRNA levels in blood in some embodiments.

FIG. 2 illustrates the Receiver-operating characteristic (ROC) curve of Formula 14 in some embodiments.

FIG. 3 illustrates comparative ROCs of three different marker selections in some embodiments.

### DETAILED DESCRIPTION OF THE DISCLOSURE

Gastrointestinal (GI) cancers, including tumors of the esophagus, stomach, pancreas, liver, bile duct, gallbladder, colon, and rectum, are one of the most frequent cancers and a leading cause of cancer deaths worldwide. According to the World Health Organization (WHO), there were an estimated 5.1 million new cases of GI cancers and 3.6 million related deaths in 2020. GI cancers account for 27% of the global cancer incidence and 36% of all cancer-related deaths. Of note, the estimated incidence and mortality rate of GI cancers are increasing, leading to a growing economic burden on patients and families. Because cancer at an early stage is considered to be more curable, early cancer identification is critical for reducing death from cancers. In addition, long-term monitoring is required for post-surgery treatment and evaluation of recurrence/relapse. Thus, improvement of current cancer detection methods is critical for providing a better diagnostic ability to counteract GI cancers.

The present disclosure is related to a method of assessing a probability of an occurrence of a cancer. In some embodiments, developing a bodily fluid-based polynucleotide molecules analysis, such as a blood-based miRNA analysis method for detecting cancers such as GI cancers. In some embodiments, a bodily fluid-based method, such as a blood-based method, which belongs to liquid biopsy test can be non-invasive, time-saving, or real-time for evaluating cancer diseases as compared with the traditional methods such as pathohistological analysis. In some embodiments, since the phenomenon of cell-free polynucleotide molecules, such as cell-free DNAs and cell-secreted miRNAs which exist in circulation have been discovered, bodily-fluid based polynucleotides, such as cell-free DNAs and blood-based miRNAs for diseases prediction and diagnosis become a rapidly growing field of study, especially in malignant tumor. However, the current method for bodily-fluid based polynucleotides detection, such as cell-free DNAs (cfDNAs) detection and blood-based miRNAs detection, in clinical practices still remains challenges in data reliability and reproducibility. Because the concentration of polynucleotides such as cfDNAs and miRNAs in the blood (aM-fM range) is relatively low, identification of the dominant and representative polynucleotide biomarkers, such as cfDNA biomarkers or miRNA biomarkers, can enhance the signal and improve the detection limitation. In some embodiments, the appropriate polynucleotide references (cfDNA or miRNA) and normalization methods for quantification of circulating polynucleotides, such as cfDNAs or miRNAs, in cancer patients are also crucial regarding the signal intensity. For the detection of bodily-fluid based polynucleotides, such as cell-free DNAs and blood-based miRNAs in GI cancer blood samples with high performance, the detection panels with the representative cancer miRNA biomarkers can be established, the appropriate polynucleotide references (cfDNA or miRNA) and the correspondent algorithms.

In some embodiments, a method for polynucleotide (cfDNA or miRNA) detection should exhibit data reliability and reproducibility. In some embodiments, processing with an appropriate normalization strategy in quantification analysis of cell-free polynucleotide-based experimental data can be of significance, due to, for example, relatively lower expression level of cell-free polynucleotide such as miRNA and cfDNA in a blood sample compared to those in a tissue sample. Due to the lower expression level, the detection limit can be considered. In some embodiments, hemolysis caused by improper specimen collection and handling process, the sample diversity and technical manipulations, the extraction methods, and the interference by inhibitors can be factors that can result in the bias of polynucleotide profiling.

Accordingly, The present disclosure is related to a method of assessing a probability of an occurrence of a cancer, in which the method comprises obtaining respective cancer-associated polynucleotide levels based on a plurality of different cancer-associated polynucleotide molecules and respective reference polynucleotide levels based on a plurality of different reference polynucleotide molecules, wherein the plurality of different cancer-associated polynucleotide molecules and the plurality of reference polynucleotide molecules may be generated based on a plurality of agents added to a solution containing a plurality of different cancer-associated miRNAs present in a bodily fluid sample and a plurality of different reference miRNAs, wherein the plurality of agents may be to interact with the plurality of different cancer-associated miRNAs and the plurality of different reference miRNAs; adjusting the respective cancer-associated polynucleotide levels based on the respective reference polynucleotide levels; assigning coefficients to the adjusted cancer-associated polynucleotide levels, respectively; and assessing the probability of the occurrence of the cancer, based on the adjusted cancer-associated polynucleotide levels and the assigned coefficients.

The present disclosure is related to a method of assessing a probability of an occurrence of a cancer, in which the method comprises adding an exogenous reference miRNA to a bodily fluid sample including a plurality of different cancer-associated miRNAs present in the bodily fluid sample; analyzing the bodily fluid sample, to determine miRNA levels of the plurality of different cancer-associated miRNAs and at least one reference miRNA level of at least one reference miRNA including the exogenous reference miRNA; adjusting at least one miRNA level among the miRNA levels based on the at least one reference miRNA level; assigning coefficients to the miRNA levels including the at least one adjusted miRNA level; and assessing the probability of the occurrence of the cancer, based on the miRNA levels and the assigned coefficients.

In some embodiments, the respective cancer-associated polynucleotide levels may indicate miRNA levels of the plurality of different cancer-associated miRNAs and respective reference polynucleotide levels may indicate reference miRNA levels of the plurality of different reference miRNAs.

In some embodiments, the method may further comprise adding the plurality of agents to the solution containing the plurality of different cancer-associated miRNAs and the plurality of different reference miRNAs.

In some embodiments, the plurality of different reference miRNAs may include an exogenous miRNA added to the bodily fluid sample. In some embodiments, the plurality of different reference miRNAs may include an endogenous miRNA. In some embodiments, the plurality of different reference miRNAs may include a plurality of endogenous miRNAs.

In some embodiments, the plurality of different cancer-associated polynucleotide molecules may include a plurality of different copy DNAs (cDNAs) amplified from a plurality of different cancer-associated miRNAs present in a bodily fluid sample.

In some embodiments, the plurality of different reference polynucleotide molecules may include a plurality of different reference copy DNAs (cDNAs) amplified from a plurality of different reference miRNAs.

In some embodiments, the cancer may be a gastrointestinal (GI) cancer.

In some embodiments, the plurality of different cancer-associated miRNAs may include hsa-miR-191-5p, hsa-miR-21-5p, hsa-miR-19a-3p, hsa-miR-145-5p, hsa-miR-23a-3p, or any combination thereof. In some embodiments, the plurality of reference miRNAs may include cel-miR-39-3p, hsa-miR-16-5p, hsa-miR-484, or any combination thereof. In some embodiments, the exogenous miRNA may include cel-miR-39-3p.

In some embodiments, the respective cancer-associated polynucleotide levels may be adjusted based on the respective reference polynucleotide levels by subtracting a reference polynucleotide level among the respective reference polynucleotide levels from a cancer-associated polynucleotide level among the respective cancer-associated polynucleotide levels, to adjust the cancer-associated polynucleotide level.

In some embodiments, the plurality of different cancer-associated miRNAs or the plurality of different reference miRNAs may include an exosomal RNA, a circulating miRNA, or a combination thereof.

In some embodiments, the probability of the occurrence of the cancer may be used in a prognostic analysis, a diagnostic analysis, or a combination thereof.

In some embodiments, the step of assessing the probability of the occurrence of the cancer may be based on Equation (1):

$$y = a 0 + a 1 * BB + a 2 * EE + a 3 * FF + a 4 * GG + a 5 * HH \qquad Equation ( 1 )$$

wherein
   y is a first probabilistic value to be based on to assess the probability of the occurrence of the cancer,
   BB, EE, FF, GG, and HH are values corresponding to the adjusted cancer-associated polynucleotide levels, and
   a0, a1, a2, a3, a4, and a5 are the assigned coefficients.

In some embodiments, BB corresponds to hsa-miR-191-5p, EE corresponds to the hsa-miR-21-5p, FF corresponds to the hsa-miR-19a-3p, GG corresponds to the hsa-miR-145-5p, and HH corresponds to the hsa-miR-23a-3p.

In some embodiments, the step of adjusting the respective cancer-associated polynucleotide levels based on the respective reference polynucleotide levels may further comprise assigning reference coefficients to the respective reference polynucleotide levels; adjusting the respective reference polynucleotide levels with the assigned reference coefficients; and adjusting the respective cancer-associated polynucleotide levels based on the adjusted respective reference polynucleotide levels.

In some embodiments, the step of assessing the probability of the occurrence of the cancer may be based on Equation (3):

$$p = exp ( y ) / ( 1 + exp ( y ) ) \qquad Equation ( 3 )$$

wherein p is a second probabilistic value to be based on to assess the probability of the occurrence of the cancer.

In some embodiments, adjusting the respective reference polynucleotide levels may be based on Equation (2):

$$α1 * AA + α2 * CC + α3 * DD \qquad Equation ( 2 )$$

wherein AA, CC, and DD are values corresponding to the respective reference polynucleotide levels, and
α1, α2, and α3 are the reference coefficients.

In some embodiments, AA corresponds to cel-miR-39-3p, CC corresponds to hsa-miR-16-5p, and DD corresponds to hsa-miR-484.

In some embodiments, the step of assessing the probability of the occurrence of the cancer may be based on Equation (3):

$$p = exp ( y ) / ( 1 + exp ( y ) ) \qquad Equation ( 3 )$$

wherein p is a second probabilistic value to be based on to assess the probability of the occurrence of the cancer.

In some embodiments, the method may further comprise, in response to the second probabilistic value exceeding a threshold value, determining that a patient from whom the bodily fluid was derived from has an elevated risk of cancer.

In some embodiments, a0 value may be from about 2.66 to about 7.03; a1 value may be 0; a2 value may be from about −1.47 to about 0.03; a3 value may be from about 2.17 to about 4.11; a4 value may be from about −1.57 to about −0.58; a5 value may be from about 0.32 to about 1.46; α1 value may be 0; α2 value may be from about −0.43 to about 0.34; and α3 value may be from about 0.59 to about 1.58.

In some embodiments, a0 value may be from about 2.66 to about 7.04; a1 value may be from about −0.44 to about 0.43; a2 value may be from about −1.47 to about 0.04; a3 value may be from about 2.14 to about 4.15; a4 value may be from about −1.58 to about −0.57; a5 value may be from about 0.26 to about 1.53; α1 value may be 0; α2 value may be from about −0.48 to about 0.38; and α3 value may be from about 0.59 to about 1.6.

In some embodiments, a0 value may be from about −0.41 to about 2.03; a1 value may be from about 0.03 to about 0.87; a2 value may be 0; a3 value may be from about 1.73 to about 3.24; a4 value may be from about −1.53 to about −0.6; a5 value may be 0; α1 value may be from about −0.91 to about −0.13; α2 value may be 0; and α3 value may be from about 0.8 to about 1.79.

In some embodiments, a0 value may be from about 0.39 to about 2.64; a1 value may be from about 0.01 to about 0.83; a2 value may be 0; a3 value may be from about 1.67 to about 3.12; a4 value may be from about −1.52 to about −0.59; a5 value may be 0; α1 value may be from about −1.01 to about −0.15; α2 value may be from about −0.62 to about 0.41; and α3 value may be from about 0.84 to about 2.02.

In some embodiments, a0 value may be from about 4.09 to about 8.49; a1 value may be 0; a2 value may be from about −0.9 to about 0.2; a3 value may be from about 1.47 to about 2.89; a4 value may be from about −1.35 to about −0.43; a5 value may be 0; α1 value may be from about −1.83 to about −0.24; α2 value may be from about −1.41 to about 0.43; and α3 value may be from about 1.22 to about 3.16.

In some embodiments, a0 value may be from about 1.96 to about 3.85; a1 value may be 0; a2 value may be 0; a3 value may be from about 1.59 to about 2.99; a4 value may be from about −1.32 to about −0.51; a5 value may be about 0; α1 value may be from about −1.09 to about −0.09; α2 value may be 0; and α3 value may be from about 0.89 to about 1.85.

In some embodiments, a0 value may be from about 2.91 to about 6.5; a1 value may be 0; a2 value may be from about −0.87 to about 0.21; a3 value may be from about 1.61 to about 3.12; a4 value may be from about −1.35 to about −0.43; a5 value may be 0; α1 value may be from about −1.36 to about −0.13; α2 value may be 0; and α3 value may be from about 0.94 to about 2.15.

In some embodiments, a0 value may be from about 3.07 to about 5.54; a1 value may be 0; a2 value may be about 0; a3 value may be from about 1.45 to about 2.74; a4 value may be from about −1.32 to about −0.51; a5 value may be 0; α1 value may be from about −1.39 to about −0.16; α2 value may be from about −1.1 to about 0.36; and α3 value may be from about 1.07 to about 2.57.

In some embodiments, a0 value may be from about 1.31 to about 3.06; a1 value may be 0; a2 value may be 0; a3 value may be from about 2 to about 3.78; a4 value may be from about −1.58 to about −0.6; a5 value may be from about 0.25 to about 1.16; α1 value may be 0; α2 value may be from about −0.39 to about 0.29; and α3 value may be from about 0.6 to about 1.46.

In some embodiments, a0 value may be from about 1.06 to about 3.31; a1 value may be from about −0.42 to about 0.43; a2 value may be 0; a3 value may be from about 1.97 to about 3.82; a4 value may be from about −1.58 to about −0.6; a5 value may be from about 0.2 to about 1.2; α1 value may be 0; α2 value may be from about −0.43 to about 0.32; and α3 value may be from about 0.59 to about 1.46.

In some embodiments, a0 value may be from about 4.47 to about 8.1; a1 value may be from about −0.05 to about 0.69; a2 value may be 0; a3 value may be from about 2 to about 3.82; a4 value may be from about −1.39 to about −0.52; a5 value may be 0; α1 value may be 0; α2 value may be from about −0.34 to about 0.44; and α3 value may be from about 0.57 to about 1.48.

In some embodiments, a0 value may be from about 5.23 to about 10.96; a1 value may be from about −0.04 to about 0.73; a2 value may be from about −0.92 to about 0.23; a3 value may be from about 2.07 to about 4; a4 value may be from about −1.4 to about −0.47; a5 value may be 0; α1 value may be 0; α2 value may be from about −0.36 to about 0.5; and α3 value may be from about 0.56 to about 1.56.

In some embodiments, a0 value may be from about 6.24 to about 12.73; a1 value may be 0; a2 value may be from about −0.74 to about 0.31; a3 value may be from about 1.83 to about 3.47; a4 value may be from about −1.24 to about −0.39; a5 value may be 0; α1 value may be 0; α2 value may be from about −0.58 to about 0.44; and α3 value may be from about 0.64 to about 1.8.

In some embodiments, a0 value may be from about 5.86 to about 10.61; a1 value may be 0; a2 value may be 0; a3 value may be from about 1.81 to about 3.38; a4 value may be from about −1.22 to about −0.45; a5 value may be 0; α1 value may be 0; α2 value may be from about −0.54 to about 0.41; and α3 value may be from about 0.65 to about 1.68.

In some embodiments, a0 value may be from about −8.04 to about −3.13; a1 value may be 0; a2 value may be from about −2.27 to about −0.62; a3 value may be from about 1.25 to about 2.59; a4 value may be 0; a5 value may be from about 0.26 to about 1.49; α1 value may be from about −1.53 to about −0.27; α2 value may be from about −1.04 to about 0.12; and α3 value may be from about 1.02 to about 2.53.

In some embodiments, a0 value may be from about −8.2 to about −2.68; a1 value may be from about −0.46 to about 0.33; a2 value may be from about −2.26 to about −0.61; a3 value may be from about 1.23 to about 2.57; a4 value may be 0; a5 value may be from about 0.26 to about 1.56; α1 value may be from about −1.57 to about −0.26; α2 value may be from about −1.15 to about 0.14; and α3 value may be from about 1.04 to about 2.61.

In some embodiments, a0 value may be from about −9.01 to about −3.7; a1 value may be 0; a2 value may be from about −2.21 to about −0.6; a3 value may be from about 1.42 to about 2.88; a4 value may be 0; a5 value may be from about 0.21 to about 1.38; α1 value may be from about −1.13 to about −0.14; α2 value may be 0; and α3 value may be from about 0.75 to about 1.76.

In some embodiments, a0 value may be from about −9.55 to about −3.35; a1 value may be from about −0.33 to about 0.5; a2 value may be from about −2.22 to about −0.6; a3 value may be from about 1.43 to about 2.88; a4 value may be 0; a5 value may be from about 0.13 to about 1.36; α1 value may be from about −1.14 to about −0.14; α2 value may be 0; and α3 value may be from about 0.72 to about 1.81.

In some embodiments, a0 value may be from about −1.89 to about 1.02; a1 value may be from about −0.51 to about 0.22; a2 value may be from about −1.7 to about −0.21; a3 value may be from about 1.56 to about 3.11; a4 value may be 0; a5 value may be from about 0.1 to about 1.25; α1 value may be 0; α2 value may be from about −0.61 to about 0.24; and α3 value may be from about 0.6 to about 1.64.

In some embodiments, a0 value may be from about −1.95 to about 0.76; a1 value may be 0; a2 value may be from about −1.71 to about −0.21; a3 value may be from about 1.64 to about 3.19; a4 value may be 0; a5 value may be from about 0.06 to about 1.1; α1 value may be 0; α2 value may be from about −0.47 to about 0.24; and α3 value may be from about 0.58 to about 1.56.

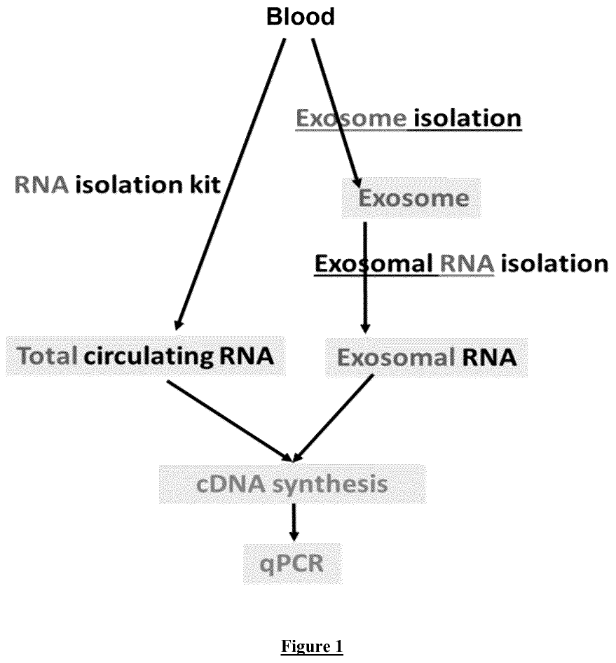

In some embodiments, cell-free polynucleotides, such as miRNA and cfDNA, can be obtained from a bodily fluid sample, such as a blood (e.g., whole blood, plasma, serum, etc.) and can be analyzed, for example by quantifying expression levels. FIG. 1 illustrates a process of analyzing miRNA levels in a blood. Referring to FIG. 1, exomal RNA level or total circulating RNA levels can be analyzed based on qPCR.

### Non-Limiting Embodiments

The present disclosure is also described by way of the following non-limiting embodiments. However, the use of these and other embodiments anywhere in the specification is illustrative only and in no way limits the scope and meaning of the disclosure. Likewise, the disclosure is not limited to any particular preferred embodiment or aspect described herein.

Indeed, modifications and variations may be apparent to those skilled in the art upon reading this specification, and such variations can be made without departing from the disclosure in spirit or in scope.

1. A method of assessing a probability of an occurrence of a cancer comprising:
obtaining
   respective cancer-associated polynucleotide levels based on a plurality of different cancer-associated polynucleotide molecules and
   respective reference polynucleotide levels based on a plurality of different reference polynucleotide molecules,
wherein the plurality of different cancer-associated polynucleotide molecules and the plurality of reference polynucleotide molecules are generated based on a plurality of agents added to a solution containing a plurality of different cancer-associated miRNAs present in a bodily fluid sample and a plurality of different reference miRNAs, wherein the plurality of agents is to interact with the plurality of different cancer-associated miRNAs and the plurality of different reference miRNAs;
adjusting the respective cancer-associated polynucleotide levels based on the respective reference polynucleotide levels;
assigning coefficients to the adjusted cancer-associated polynucleotide levels, respectively; and
assessing the probability of the occurrence of the cancer, based on the adjusted cancer-associated polynucleotide levels and the assigned coefficients,
wherein the assessed probability based on the assigned coefficients exhibits at least 0.9 of an area under the receiver operating characteristic (ROC) curve (AUC); or
a method of preparing adjusted cancer-associated polynucleotide levels useful for assessing a probability of an occurrence of a cancer comprising:
obtaining
   respective cancer-associated polynucleotide levels based on a plurality of different cancer-associated polynucleotide molecules and
   respective reference polynucleotide levels based on a plurality of different reference polynucleotide molecules,
wherein the plurality of different cancer-associated polynucleotide molecules and the plurality of reference polynucleotide molecules are generated based on a plurality of agents added to a solution containing a plurality of different cancer-associated miRNAs present in a bodily fluid sample and a plurality of different reference miRNAs, wherein the plurality of agents is to interact with the plurality of different cancer-associated miRNAs and the plurality of different reference miRNAs;
adjusting the respective cancer-associated polynucleotide levels based on the respective reference polynucleotide levels;
assigning coefficients to the adjusted cancer-associated polynucleotide levels, respectively; and
assessing the probability of the occurrence of the cancer, based on the adjusted cancer-associated polynucleotide levels and the assigned coefficients,
wherein the assessed probability based on the assigned coefficients exhibits at least 0.9 of an area under the receiver operating characteristic (ROC) curve (AUC).

2. The method of Embodiment 1, wherein the respective cancer-associated polynucleotide levels indicate miRNA levels of the plurality of different cancer-associated miRNAs and respective reference polynucleotide levels indicate reference miRNA levels of the plurality of different reference miRNAs.

3. The method of Embodiment 1-2, further comprising adding the plurality of agents to the solution containing the plurality of different cancer-associated miRNAs and the plurality of different reference miRNAs.

4. The method of Embodiment 1-3, wherein the plurality of different reference miRNAs includes an exogenous miRNA added to the bodily fluid sample.

5. The method of Embodiment 1-4, wherein the plurality of different reference miRNAs includes an endogenous miRNA.

6. The method of Embodiment 1-5, wherein the plurality of different reference miRNAs includes a plurality of endogenous miRNAs.

7. The method of Embodiment 1-6, wherein the plurality of different cancer-associated polynucleotide molecules includes a plurality of different copy DNAs (cDNAs) amplified from a plurality of different cancer-associated miRNAs present in a bodily fluid sample.

8. The method of Embodiment 1-7, wherein the plurality of different reference polynucleotide molecules includes a plurality of different reference copy DNAs (cDNAs) amplified from a plurality of different reference miRNAs.

9. The method of Embodiment 1-8, wherein the cancer is a gastrointestinal (GI) cancer.

10. The method of Embodiment 1-9, wherein the plurality of different cancer-associated miRNAs includes hsa-miR-191-5p, hsa-miR-21-5p, hsa-miR-19a-3p, hsa-miR-145-5p, hsa-miR-23a-3p, or any combination thereof.

11. The method of Embodiment 1-10, wherein the plurality of reference miRNAs includes cel-miR-39-3p, hsa-miR-16-5p, hsa-miR-484, or any combination thereof.

12. The method of Embodiment 1-4, wherein the exogenous miRNA includes cel-miR-39-3p.

13. The method of Embodiment 1-12, wherein the respective cancer-associated polynucleotide levels are adjusted based on the respective reference polynucleotide levels by subtracting a reference polynucleotide level among the respective reference polynucleotide levels from a cancer-associated polynucleotide level among the respective cancer-associated polynucleotide levels, to adjust the cancer-associated polynucleotide level.

14. The method of Embodiment 1-13, wherein the plurality of different cancer-associated miRNAs or the plurality of different reference miRNAs include an exosomal RNA, a circulating miRNA, or a combination thereof.

15. The method of Embodiment 1-14, wherein the probability of the occurrence of the cancer is used in a prognostic analysis, a diagnostic analysis, or a combination thereof.

16. The method of Embodiment 1-15, wherein the step of assessing the probability of the occurrence of the cancer is based on Equation (1):

$$y = a 0 + a 1 * BB + a 2 * EE + a 3 * FF + a 4 * GG + a 5 * HH \qquad Equation ( 1 )$$

wherein
y is a first probabilistic value to be based on to assess the probability of the occurrence of the cancer,
BB, EE, FF, GG, and HH are values corresponding to the adjusted cancer-associated polynucleotide levels, and
a0, a1, a2, a3, a4, and a5 are the assigned coefficients.

17. The method of Embodiment 16, wherein BB corresponds to hsa-miR-191-5p, EE corresponds to the hsa-miR-21-5p, FF corresponds to the hsa-miR-19a-3p, GG corresponds to the hsa-miR-145-5p, and HH corresponds to the hsa-miR-23a-3p.

18. The method of Embodiment 1-17, wherein the step of adjusting the respective cancer-associated polynucleotide levels based on the respective reference polynucleotide levels further comprises:
assigning reference coefficients to the respective reference polynucleotide levels;
adjusting the respective reference polynucleotide levels with the assigned reference coefficients; and
adjusting the respective cancer-associated polynucleotide levels based on the adjusted respective reference polynucleotide levels.

19. The method of Embodiment 16-18, wherein the step of assessing the probability of the occurrence of the cancer is based on Equation (3):

$$p = exp ( y ) / ( 1 + exp ( y ) ) \qquad Equation ( 3 )$$

wherein p is a second probabilistic value to be based on to assess the probability of the occurrence of the cancer.

20. The method of Embodiment 18, wherein adjusting the respective reference polynucleotide levels is based on Equation (2):

$$α1 * AA + α2 * CC + α3 * DD \qquad Equation ( 2 )$$

wherein AA, CC, and DD are values corresponding to the respective reference polynucleotide levels, and
α1, α2, and α3 are the reference coefficients.

21. The method of Embodiment 20, wherein AA corresponds to cel-miR-39-3p, CC corresponds to hsa-miR-16-5p, and DD corresponds to hsa-miR-484.

22. The method of Embodiment 1-15, wherein the step of assessing the probability of the occurrence of the cancer is based on Equation (4):

$$y = a 0 + a 1 * [ BB - ( α1 * AA + α2 * CC + α3 * DD ) ] + a 2 * [ EE - ( α 1 * AA + α 2 * CC + α3 * DD ) ] + a 3 * [ FF - ( α1 * AA + α2 * CC + α3 * DD ) ] + a 4 * [ GG - ( α1 * AA + α2 * CC + α3 * DD ) ] + a 5 * [ HH - ( α1 * AA + α2 * CC + α3 * DD ) ] \qquad Equation ( 4 )$$

wherein
y is a first probabilistic value to be based on to assess the probability of the occurrence of the cancer,
BB, EE, FF, GG, and HH are values corresponding to the respective cancer-associated polynucleotide levels,
a0, a1, a2, a3, a4, and a5 are the assigned coefficients,
AA, CC, and DD are values corresponding to the respective reference polynucleotide levels, and
α1, α2, and α3 are the reference coefficients.

23. The method of Embodiment 22, wherein AA corresponds to cel-miR-39-3p, CC corresponds to hsa-miR-16-5p, and DD corresponds to hsa-miR-484.

24. The method of Embodiment 20-23, wherein the step of assessing the probability of the occurrence of the cancer is based on Equation (3):

$$p = exp ( y ) / ( 1 + exp ( y ) ) \qquad Equation ( 3 )$$

wherein p is a second probabilistic value to be based on to assess the probability of the occurrence of the cancer.

25. The method of Embodiment 16-24, wherein, the method further comprises, in response to the second probabilistic value exceeding a threshold value, determining that a patient from whom the bodily fluid was derived from has an elevated risk of cancer.

26. The method of Embodiment 16-24, wherein, the method further comprises, in response to the second probabilistic value exceeding a threshold value, determining that a patient from whom the bodily fluid was derived from has an elevated risk of cancer.

27. The method of Embodiment 20-24, wherein
a0 value is from about 2.66 to about 7.03;
a1 value is 0;
a2 value is from about −1.47 to about 0.03;
a3 value is from about 2.17 to about 4.11;
a4 value is from about −1.57 to about −0.58;
a5 value is from about 0.32 to about 1.46;
α1 value is 0;
α2 value is from about −0.43 to about 0.34; and
α3 value is from about 0.59 to about 1.58.

28. The method of Embodiment 20-24, wherein
a0 value is from about 2.66 to about 7.04;
a1 value is from about −0.44 to about 0.43;
a2 value is from about −1.47 to about 0.04;
a3 value is from about 2.14 to about 4.15;
a4 value is from about −1.58 to about −0.57;
a5 value is from about 0.26 to about 1.53;
α1 value is 0;
α2 value is from about −0.48 to about 0.38; and
α3 value is from about 0.59 to about 1.6.

29. The method of Embodiment 20-24, wherein
a0 value is from about −0.41 to about 2.03;
a1 value is from about 0.03 to about 0.87;
a2 value is 0;
a3 value is from about 1.73 to about 3.24;
a4 value is from about −1.53 to about −0.6;
a5 value is 0;
α1 value is from about −0.91 to about −0.13;
α2 value is 0; and
α3 value is from about 0.8 to about 1.79.

30. The method of Embodiment 20-24, wherein
a0 value is from about 0.39 to about 2.64;
a1 value is from about 0.01 to about 0.83;
a2 value is 0;
a3 value is from about 1.67 to about 3.12;
a4 value is from about −1.52 to about −0.59;
a5 value is 0;
α1 value is from about −1.01 to about −0.15;
α2 value is from about −0.62 to about 0.41; and
α3 value is from about 0.84 to about 2.02.

31. The method of Embodiment 20-24, wherein
a0 value is from about 4.09 to about 8.49;
a1 value is 0;
a2 value is from about −0.9 to about 0.2;
a3 value is from about 1.47 to about 2.89;
a4 value is from about −1.35 to about −0.43;
a5 value is 0;
α1 value is from about −1.83 to about −0.24;
α2 value is from about −1.41 to about 0.43; and
α3 value is from about 1.22 to about 3.16.

32. The method of Embodiment 20-24, wherein
a0 value is from about 1.96 to about 3.85;
a1 value is 0;
a2 value is 0;
a3 value is from about 1.59 to about 2.99;
a4 value is from about −1.32 to about −0.51;
a5 value is about 0;
α1 value is from about −1.09 to about −0.09;
α2 value is 0; and
α3 value is from about 0.89 to about 1.85.

33. The method of Embodiment 20-24, wherein
a0 value is from about 2.91 to about 6.5;
a1 value is 0;
a2 value is from about −0.87 to about 0.21;
a3 value is from about 1.61 to about 3.12;
a4 value is from about −1.35 to about −0.43;
a5 value is 0;
α1 value is from about −1.36 to about −0.13;
α2 value is 0; and
α3 value is from about 0.94 to about 2.15.

34. The method of Embodiment 20-24, wherein
a0 value is from about 3.07 to about 5.54;
a1 value is 0;
a2 value is about 0;
a3 value is from about 1.45 to about 2.74;
a4 value is from about −1.32 to about −0.51;
a5 value is 0;
α1 value is from about −1.39 to about −0.16;
α2 value is from about −1.1 to about 0.36; and
α3 value is from about 1.07 to about 2.57.

35. The method of Embodiment 20-24, wherein
a0 value is from about 1.31 to about 3.06;
a1 value is 0;
a2 value is 0;
a3 value is from about 2 to about 3.78;
a4 value is from about −1.58 to about −0.6;
a5 value is from about 0.25 to about 1.16;
α1 value is 0;
α2 value is from about −0.39 to about 0.29; and
α3 value is from about 0.6 to about 1.46.

36. The method of Embodiment 20-24, wherein
a0 value is from about 1.06 to about 3.31;
a1 value is from about −0.42 to about 0.43;
a2 value is 0;
a3 value is from about 1.97 to about 3.82;
a4 value is from about −1.58 to about −0.6;
a5 value is from about 0.2 to about 1.2;
α1 value is 0;
α2 value is from about −0.43 to about 0.32; and
α3 value is from about 0.59 to about 1.46.

37. The method of Embodiment 20-24, wherein
a0 value is from about 4.47 to about 8.1;
a1 value is from about −0.05 to about 0.69;
a2 value is 0;
a3 value is from about 2 to about 3.82;
a4 value is from about −1.39 to about −0.52;
a5 value is 0;
α1 value is 0;
α2 value is from about −0.34 to about 0.44; and
α3 value is from about 0.57 to about 1.48.

38. The method of Embodiment 20-24, wherein
a0 value is from about 5.23 to about 10.96;
a1 value is from about −0.04 to about 0.73;
a2 value is from about −0.92 to about 0.23;
a3 value is from about 2.07 to about 4;
a4 value is from about −1.4 to about −0.47;
a5 value is 0;
α1 value is 0;
α2 value is from about −0.36 to about 0.5; and
α3 value is from about 0.56 to about 1.56.

39. The method of Embodiment 20-24, wherein
a0 value is from about 6.24 to about 12.73;
a1 value is 0;
a2 value is from about −0.74 to about 0.31;
a3 value is from about 1.83 to about 3.47;
a4 value is from about −1.24 to about −0.39;
a5 value is 0;
α1 value is 0;
α2 value is from about −0.58 to about 0.44; and
α3 value is from about 0.64 to about 1.8.

40. The method of Embodiment 20-24, wherein
a0 value is from about 5.86 to about 10.61;
a1 value is 0;
a2 value is 0;
a3 value is from about 1.81 to about 3.38;
a4 value is from about −1.22 to about −0.45;
a5 value is 0;
α1 value is 0;
α2 value is from about −0.54 to about 0.41; and
α3 value is from about 0.65 to about 1.68.

41. The method of Embodiment 20-24, wherein
a0 value is from about −8.04 to about −3.13;
a1 value is 0;
a2 value is from about −2.27 to about −0.62;
a3 value is from about 1.25 to about 2.59;
a4 value is 0;
a5 value is from about 0.26 to about 1.49;
α1 value is from about −1.53 to about −0.27;
α2 value is from about −1.04 to about 0.12; and
α3 value is from about 1.02 to about 2.53.

42. The method of Embodiment 20-24, wherein
a0 value is from about −8.2 to about −2.68;
a1 value is from about −0.46 to about 0.33;
a2 value is from about −2.26 to about −0.61;
a3 value is from about 1.23 to about 2.57;
a4 value is 0;
a5 value is from about 0.26 to about 1.56;
α1 value is from about −1.57 to about −0.26;
α2 value is from about −1.15 to about 0.14; and
α3 value is from about 1.04 to about 2.61.

43. The method of Embodiment 20-24, wherein
a0 value is from about −9.01 to about −3.7;
a1 value is 0;
a2 value is from about −2.21 to about −0.6;
a3 value is from about 1.42 to about 2.88;
a4 value is 0;
a5 value is from about 0.21 to about 1.38;
α1 value is from about −1.13 to about −0.14;
α2 value is 0; and
α3 value is from about 0.75 to about 1.76.

44. The method of Embodiment 20-24, wherein
a0 value is from about −9.55 to about −3.35;
a1 value is from about −0.33 to about 0.5;
a2 value is from about −2.22 to about −0.6;
a3 value is from about 1.43 to about 2.88;
a4 value is 0;
a5 value is from about 0.13 to about 1.36;
α1 value is from about −1.14 to about −0.14;
α2 value is 0; and
α3 value is from about 0.72 to about 1.81.

45. The method of Embodiment 20-24, wherein
a0 value is from about −1.89 to about 1.02;
a1 value is from about −0.51 to about 0.22;
a2 value is from about −1.7 to about −0.21;
a3 value is from about 1.56 to about 3.11;
a4 value is 0;
a5 value is from about 0.1 to about 1.25;
α1 value is 0;
α2 value is from about −0.61 to about 0.24; and
α3 value is from about 0.6 to about 1.64.

46. The method of Embodiment 20-24, wherein
a0 value is from about −1.95 to about 0.76;
a1 value is 0;
a2 value is from about −1.71 to about −0.21;
a3 value is from about 1.64 to about 3.19;
a4 value is 0;
a5 value is from about 0.06 to about 1.1;
α1 value is 0;
α2 value is from about −0.47 to about 0.24; and
α3 value is from about 0.58 to about 1.56.

47. A method of assessing a probability of an occurrence of a cancer comprising:
adding an exogenous reference miRNA to a bodily fluid sample including a plurality of different cancer-associated miRNAs present in the bodily fluid sample;
analyzing the bodily fluid sample, to determine miRNA levels of the plurality of different cancer-associated miRNAs and at least one reference miRNA level of at least one reference miRNA including the exogenous reference miRNA;
adjusting at least one miRNA level among the miRNA levels based on the at least one reference miRNA level;
assigning coefficients to the miRNA levels including the at least one adjusted miRNA level; and
assessing the probability of the occurrence of the cancer, based on the miRNA levels and the assigned coefficients,
wherein the assessed probability based on the assigned coefficients exhibits at least 0.9 of an area under the receiver operating characteristic (ROC) curve (AUC), or
a method of preparing at least one adjusted miRNA level useful for assessing a probability of an occurrence of a cancer comprising:
adding an exogenous reference miRNA to a bodily fluid sample including a plurality of different cancer-associated miRNAs present in the bodily fluid sample;
analyzing the bodily fluid sample, to determine miRNA levels of the plurality of different cancer-associated miRNAs and at least one reference miRNA level of at least one reference miRNA including the exogenous reference miRNA;
adjusting at least one miRNA level among the miRNA levels based on the at least one reference miRNA level;
assigning coefficients to the miRNA levels including the at least one adjusted miRNA level; and
assessing the probability of the occurrence of the cancer, based on the miRNA levels and the assigned coefficients,
wherein the assessed probability based on the assigned coefficients exhibits at least 0.9 of an area under the receiver operating characteristic (ROC) curve (AUC).

48. The method of Embodiment 47, wherein the at least one reference miRNA includes a plurality of different reference miRNAs, and
the method further comprises:
analyzing the bodily fluid sample including the exogenous miRNA, to determine reference miRNA levels of the plurality of different reference miRNAs;
adjusting corresponding miRNA levels among the miRNA levels based on the reference miRNA levels;
assigning coefficients to the miRNA levels including the adjusted corresponding miRNA levels; and
assessing the probability of the occurrence of the cancer, based on the miRNA levels and the assigned coefficients.

49. The method of Embodiment 47-48, wherein the plurality of different reference miRNAs includes an endogenous miRNA.

50. The method of Embodiment 47-49, wherein the plurality of different reference miRNAs includes a plurality of endogenous miRNAs.

51. The method of Embodiment 47-50, wherein the miRNA levels of the plurality of different cancer-associated miRNAs are determined based on a plurality of different copy DNAs (cDNAs) amplified from the plurality of different cancer-associated miRNAs.

52. The method of Embodiment 47-51, wherein the reference miRNA level of the reference miRNA a reference copy DNA (cDNA) amplified from the reference miRNA.

53. The method of Embodiment 47-52, wherein the cancer is a gastrointestinal (GI) cancer.

54. The method of Embodiment 47-53, wherein the plurality of different cancer-associated miRNAs includes hsa-miR-191-5p, hsa-miR-21-5p, hsa-miR-19a-3p, hsa-miR-145-5p, hsa-miR-23a-3p, or any combination thereof.

55. The method of Embodiment 47-54, wherein the at least one reference miRNA includes cel-miR-39-3p, hsa-miR-16-5p, hsa-miR-484, or any combination thereof.

56. The method of Embodiment 47-55, wherein the respective cancer-associated miRNA levels are adjusted based on the respective reference miRNA levels by subtracting a reference miRNA level among the respective reference miRNA levels from a cancer-associated miRNA level among the respective cancer-associated miRNA levels, to adjust the cancer-associated miRNA level.

57. The method of Embodiment 47-56, wherein the plurality of different cancer-associated miRNAs or the plurality of different reference miRNAs include an exosomal RNA, a circulating miRNA, or a combination thereof.

58. The method of Embodiment 47-57, wherein the probability of the occurrence of the cancer is used in a prognostic analysis, a diagnostic analysis, or a combination thereof.

59. The method of Embodiment 47-58, wherein the step of assessing the probability of the occurrence of the cancer is based on Equation (1):

$$y = a 0 + a 1 * BB + a 2 * EE + a 3 * FF + a 4 * GG + a 5 * HH \qquad Equation ( 1 )$$

wherein
y is a first probabilistic value to be based on to assess the probability of the occurrence of the cancer,
BB, EE, FF, GG, and HH are values corresponding to the adjusted cancer-associated miRNA levels,
and a0, a1, a2, a3, a4, and a5 are the assigned coefficients.

60. The method of Embodiment 59, wherein BB corresponds to hsa-miR-191-5p, EE corresponds to the hsa-miR-21-5p, FF corresponds to the hsa-miR-19a-3p, GG corresponds to the hsa-miR-145-5p, and HH corresponds to the hsa-miR-23a-3p.

61. The method of Embodiment 47-60, wherein the step of adjusting the respective cancer-associated miRNA levels based on the respective reference miRNA levels further comprises:
assigning reference coefficients to the respective reference miRNA levels;
adjusting the respective reference miRNA levels with the assigned reference coefficients; and
adjusting the respective cancer-associated miRNA levels based on the adjusted respective reference miRNA levels.

62. The method of Embodiment 59-61, wherein the step of assessing the probability of the occurrence of the cancer is based on Equation (3):

$$p = exp ( y ) / ( 1 + exp ( y ) ) \qquad Equation ( 3 )$$

wherein p is a second probabilistic value to be based on to assess the probability of the occurrence of the cancer.

63. The method of Embodiment 61, wherein adjusting the respective reference polynucleotide levels is based on Equation (2):

$$α1 * AA + α2 * CC + α3 * DD \qquad Equation ( 2 )$$

wherein AA, CC, and DD are values corresponding to the respective reference polynucleotide levels, and
α1, α2, and α3 are the reference coefficients.

64. The method of Embodiment 63, wherein AA corresponds to cel-miR-39-3p, CC corresponds to hsa-miR-16-5p, and DD corresponds to hsa-miR-484.

65. The method of Embodiment 47-58, wherein the step of assessing the probability of the occurrence of the cancer is based on Equation (4):

$$y = a 0 + a 1 * [ BB - ( α1 * AA + α2 * CC + α3 * DD ) ] + a 2 * [ EE - ( α 1 * AA + α 2 * CC + α3 * DD ) ] + a 3 * [ FF - ( α1 * AA + α2 * CC + α3 * DD ) ] + a 4 * [ GG - ( α1 * AA + α2 * CC + α3 * DD ) ] + a 5 * [ HH - ( α1 * AA + α2 * CC + α3 * DD ) ] \qquad Equation ( 4 )$$

wherein
y is a first probabilistic value to be based on to assess the probability of the occurrence of the cancer,
BB, EE, FF, GG, and HH are values corresponding to the respective cancer-associated polynucleotide levels,
a0, a1, a2, a3, a4, and a5 are the assigned coefficients,
AA, CC, and DD are values corresponding to the respective reference polynucleotide levels, and
α1, α2, and α3 are the reference coefficients.

66. The method of Embodiment 65, wherein AA corresponds to cel-miR-39-3p, CC corresponds to hsa-miR-16-5p, and DD corresponds to hsa-miR-484.

67. The method of Embodiment 63-66, wherein the step of assessing the probability of the occurrence of the cancer is based on Equation (3):

$$p = exp ( y ) / ( 1 + exp ( y ) ) \qquad Equation ( 3 )$$

wherein p is a second probabilistic value to be based on to assess the probability of the occurrence of the cancer.

68. The method of Embodiment 62, wherein, the method further comprises, in response to the second probabilistic value exceeding a threshold value, determining that a patient from whom the bodily fluid was derived from has an elevated risk of cancer.

69. The method of Embodiment 62, wherein, the method further comprises, in response to the second probabilistic value exceeding a threshold value, determining that a patient from whom the bodily fluid was derived from has an elevated risk of cancer.

70. The method of Embodiment 63-67, wherein
a0 value is from about 2.66 to about 7.03;
a1 value is 0;
a2 value is from about −1.47 to about 0.03;
a3 value is from about 2.17 to about 4.11;
a4 value is from about −1.57 to about −0.58;
a5 value is from about 0.32 to about 1.46;
α1 value is 0;
α2 value is from about −0.43 to about 0.34; and
α3 value is from about 0.59 to about 1.58.

71. The method of Embodiment 63-67, wherein
a0 value is from about 2.66 to about 7.04;
a1 value is from about −0.44 to about 0.43;
a2 value is from about −1.47 to about 0.04;
a3 value is from about 2.14 to about 4.15;
a4 value is from about −1.58 to about −0.57;
a5 value is from about 0.26 to about 1.53;
α1 value is 0;
α2 value is from about −0.48 to about 0.38; and
α3 value is from about 0.59 to about 1.6.

72. The method of Embodiment 63-67, wherein
a0 value is from about −0.41 to about 2.03;
a1 value is from about 0.03 to about 0.87;
a2 value is 0;
a3 value is from about 1.73 to about 3.24;
a4 value is from about −1.53 to about −0.6;
a5 value is 0;
α1 value is from about −0.91 to about −0.13;
α2 value is 0; and
α3 value is from about 0.8 to about 1.79.

73. The method of Embodiment 63-67, wherein
a0 value is from about 0.39 to about 2.64;
a1 value is from about 0.01 to about 0.83;
a2 value is 0;
a3 value is from about 1.67 to about 3.12;
a4 value is from about −1.52 to about −0.59;
a5 value is 0;
α1 value is from about −1.01 to about −0.15;
α2 value is from about −0.62 to about 0.41; and
α3 value is from about 0.84 to about 2.02.

74. The method of Embodiment 63-67, wherein
a0 value is from about 4.09 to about 8.49;
a1 value is 0;
a2 value is from about −0.9 to about 0.2;
a3 value is from about 1.47 to about 2.89;
a4 value is from about −1.35 to about −0.43;
a5 value is 0;
α1 value is from about −1.83 to about −0.24;
α2 value is from about −1.41 to about 0.43; and
α3 value is from about 1.22 to about 3.16.

75. The method of Embodiment 63-67, wherein
a0 value is from about 1.96 to about 3.85;
a1 value is 0;
a2 value is 0;
a3 value is from about 1.59 to about 2.99;
a4 value is from about −1.32 to about −0.51;
a5 value is about 0;
α1 value is from about −1.09 to about −0.09;
α2 value is 0; and
α3 value is from about 0.89 to about 1.85.

76. The method of Embodiment 63-67, wherein
a0 value is from about 2.91 to about 6.5;
a1 value is 0;
a2 value is from about −0.87 to about 0.21;
a3 value is from about 1.61 to about 3.12;
a4 value is from about −1.35 to about −0.43;
a5 value is 0;
α1 value is from about −1.36 to about −0.13;
α2 value is 0; and
α3 value is from about 0.94 to about 2.15.

77. The method of Embodiment 63-67, wherein
a0 value is from about 3.07 to about 5.54;
a1 value is 0;
a2 value is about 0;
a3 value is from about 1.45 to about 2.74;
a4 value is from about −1.32 to about −0.51;
a5 value is 0;
α1 value is from about −1.39 to about −0.16;
α2 value is from about −1.1 to about 0.36; and
α3 value is from about 1.07 to about 2.57.

78. The method of Embodiment 63-67, wherein
a0 value is from about 1.31 to about 3.06;
a1 value is 0;
a2 value is 0;
a3 value is from about 2 to about 3.78;
a4 value is from about −1.58 to about −0.6;
a5 value is from about 0.25 to about 1.16;
α1 value is 0;
α2 value is from about −0.39 to about 0.29; and
α3 value is from about 0.6 to about 1.46.

79. The method of Embodiment 63-67, wherein
a0 value is from about 1.06 to about 3.31;
a1 value is from about −0.42 to about 0.43;
a2 value is 0;
a3 value is from about 1.97 to about 3.82;
a4 value is from about −1.58 to about −0.6;
a5 value is from about 0.2 to about 1.2;
α1 value is 0;
α2 value is from about −0.43 to about 0.32; and
α3 value is from about 0.59 to about 1.46.

80. The method of Embodiment 63-67, wherein
a0 value is from about 4.47 to about 8.1;
a1 value is from about −0.05 to about 0.69;
a2 value is 0;
a3 value is from about 2 to about 3.82;
a4 value is from about −1.39 to about −0.52;
a5 value is 0;
α1 value is 0;
α2 value is from about −0.34 to about 0.44; and
α3 value is from about 0.57 to about 1.48.

81. The method of Embodiment 63-67, wherein
a0 value is from about 5.23 to about 10.96;
a1 value is from about −0.04 to about 0.73;
a2 value is from about −0.92 to about 0.23;
a3 value is from about 2.07 to about 4;
a4 value is from about −1.4 to about −0.47;
a5 value is 0;
α1 value is 0;
α2 value is from about −0.36 to about 0.5; and
α3 value is from about 0.56 to about 1.56.

82. The method of Embodiment 63-67, wherein
a0 value is from about 6.24 to about 12.73;
a1 value is 0;
a2 value is from about −0.74 to about 0.31;
a3 value is from about 1.83 to about 3.47;
a4 value is from about −1.24 to about −0.39;
a5 value is 0;
α1 value is 0;
α2 value is from about −0.58 to about 0.44; and
α3 value is from about 0.64 to about 1.8.

83. The method of Embodiment 63-67, wherein
a0 value is from about 5.86 to about 10.61;
a1 value is 0;
a2 value is 0;
a3 value is from about 1.81 to about 3.38;
a4 value is from about −1.22 to about −0.45;
a5 value is 0;
α1 value is 0;
α2 value is from about −0.54 to about 0.41; and
α3 value is from about 0.65 to about 1.68.

84. The method of Embodiment 63-67, wherein
a0 value is from about −8.04 to about −3.13;
a1 value is 0;
a2 value is from about −2.27 to about −0.62;
a3 value is from about 1.25 to about 2.59;
a4 value is 0;
a5 value is from about 0.26 to about 1.49;
α1 value is from about −1.53 to about −0.27;
α2 value is from about −1.04 to about 0.12; and
α3 value is from about 1.02 to about 2.53.

85. The method of Embodiment 63-67, wherein
a0 value is from about −8.2 to about −2.68;
a1 value is from about −0.46 to about 0.33;
a2 value is from about −2.26 to about −0.61;
a3 value is from about 1.23 to about 2.57;
a4 value is 0;
a5 value is from about 0.26 to about 1.56;
α1 value is from about −1.57 to about −0.26;
α2 value is from about −1.15 to about 0.14; and
α3 value is from about 1.04 to about 2.61.

86. The method of Embodiment 63-67, wherein
a0 value is from about −9.01 to about −3.7;
a1 value is 0;
a2 value is from about −2.21 to about −0.6;
a3 value is from about 1.42 to about 2.88;
a4 value is 0;
a5 value is from about 0.21 to about 1.38;
α1 value is from about −1.13 to about −0.14;
α2 value is 0; and
α3 value is from about 0.75 to about 1.76.

87. The method of Embodiment 63-67, wherein
a0 value is from about −9.55 to about −3.35;
a1 value is from about −0.33 to about 0.5;
a2 value is from about −2.22 to about −0.6;
a3 value is from about 1.43 to about 2.88;
a4 value is 0;
a5 value is from about 0.13 to about 1.36;
α1 value is from about −1.14 to about −0.14;
α2 value is 0; and
α3 value is from about 0.72 to about 1.81.

88. The method of Embodiment 63-67, wherein
a0 value is from about −1.89 to about 1.02;
a1 value is from about −0.51 to about 0.22;
a2 value is from about −1.7 to about −0.21;
a3 value is from about 1.56 to about 3.11;
a4 value is 0;
a5 value is from about 0.1 to about 1.25;
α1 value is 0;
α2 value is from about −0.61 to about 0.24; and
α3 value is from about 0.6 to about 1.64.

89. The method of Embodiment 63-67, wherein
a0 value is from about −1.95 to about 0.76;
a1 value is 0;
a2 value is from about −1.71 to about −0.21;
a3 value is from about 1.64 to about 3.19;
a4 value is 0;
a5 value is from about 0.06 to about 1.1;
α1 value is 0;
α2 value is from about −0.47 to about 0.24; and
α3 value is from about 0.58 to about 1.56.

### Non-Limiting Examples

The following examples are provided to illustrate selected embodiments. They should not be considered as limiting the scope of the invention, but merely as being illustrative and representative thereof. Thus, the examples provided below, while illustrated with a particular medical device or active agent, are applicable to the range of medical devices and active agents described herein.

### Example 1: Development of a Detection Method

To develop an optimal method for evaluation of miRNAs abundant in GI cancer blood samples, a dataset composed of 160 subjects with GI cancers (cancer group) and 74 subjects without cancer (non-cancer group) was established (Table 1). The cancer group included plasma derived from the subjects with gastric cancer (n=34), colon cancer (n=25), pancreatic cancer (n=24) and hepatocellular carcinoma (n=30) and the serum derived from the subjects with pancreatic cancer (n=27). The non-cancer group recruited the plasma derived from the subjects with benign hyperplasia (n=24) and the serum derived from the healthy subjects (n=22). The plasma and serum samples were stored at −80° C. before use.

TABLE 1
Description of the dataset

| | |
|---|---|
| Group | Specimen (n) |

GI Cancer

| | |
|---|---|
| Gastric cancer | Plasma (34) |
| Colon cancer | Plasma (25) |
| Pancreatic cancer | Plasma (24), Serum (27) |
| Hepatocellular carcinoma | Plasma (30) |

Non-cancer

| | |
|---|---|
| Benign hyperplasia | Plasma (24) |
| Healthy | Serum (22) |

miRNA Biomarkers and References for Detection of GI Cancers

According to a computational analysis of published cancer miRNA datasets and literature-based selection, the miRNA gene BB, EE, FF, GG, HH were chosen as the representative miRNA biomarkers for detection of GI cancers as follows.

### Detection Targets

| Name | Reference/Biomarker | miRNA ID |
|---|---|---|
| AA | Reference | cel-miR-39-3p |
| BB | Biomarker | hsa-miR-191-5p |
| CC | Reference | hsa-miR-16-5p |
| DD | Reference | hsa-miR-484 |
| EE | Biomarker | hsa-miR-21-5p |
| FF | Biomarker | hsa-miR-19a-3p |
| GG | Biomarker | hsa-miR-145-5p |
| HH | Biomarker | hsa-miR-23a-3p |

For qPCR normalization, the exogenous miRNA reference AA was added into blood (e.g., whole blood, plasma, serum, etc.) sample during miRNA extraction. In addition, the endogenous miRNA reference CC and DD were also chosen for qPCR normalization. The abundance of the selected miRNA biomarkers and references was examined by qPCR, and the obtained threshold cycle (Ct) value of each miRNA was subsequently used for statistical analysis.

### Algorithms for Detection of GI Cancers

The algorithms for detection of GI cancers were developed corresponding to the diverse compositions of biomarkers (BB, EE, FF, GG and HH) and references (AA, CC and DD). In consideration of the selected miRNA biomarkers and references, an occurrence probability for detection of GI cancers was defined:

$$Occurrence probability = exp ( y ) / [ 1 + exp ( y ) ] ,$$

$$where$$

$$y = a 0 + a 1 * [ BB - ( α1 * AA + α2 * CC + α3 * DD ) ] + a 2 * [ EE - ( α 1 * AA + α 2 * CC + α3 * DD ) ] + a 3 * [ FF - ( α1 * AA + α2 * CC + α3 * DD ) ] + a 4 * [ GG - ( α1 * AA + α2 * CC + α3 * DD ) ] + a 5 * [ HH - ( α1 * AA + α2 * CC + α3 * DD ) ]$$

Theoretically, a total of 255 compositions of formulas could be established for diverse compositions of the miRNA biomarker BB, EE, FF, GG and HH, and the miRNA reference AA, CC and DD. The formulas were subsequently evaluated by statistical analysis according to the dataset.

### Statistical Analysis

The occurrence probability was assessed by calculated by inserting the qPCR data of AA, BB, CC, DD, EE, FF, GG and HH into the formula. The ROC (receiver operating characteristic) curves were generated by plotting sensitivity against 1-specificity for each possible occurrence probability thresholds, and an area under the ROC curve (AUC) was then calculated to show the performance of the detection panels. Here, the panels are a composition of biomarkers, references and algorithm in detection of GI cancers. Furthermore, the classifier performance analysis was performed to show the proportion of correct assignment of cancer under the occurrence probability threshold 50%. Here, Classifier is a detection panel with fixed threshold for division of two populations such as GI cancer and non-GI cancer populations.

### The Performance of Formulas in Detection of GI Cancers

By a statistical analysis using the established dataset, the formulas ranked by AUC values in detection of GI cancers were chosen (Table 2). The ROC curves and coefficients of these formulas were shown in t Table 3. All of these formulas showed high AUC values (>0.8), revealed the excellent performance of these formulas in detection of GI cancers. The classifier performances analysis demonstrated that over 90.0% sample from subjects with GI cancers can correctly detect GI cancers by the present formulas (Table 2).

For example, the following is an example calculation of Formula 33 in Table 2.

$$Formula 033 ( Reference : AA , CC and DD ) . \qquad Formula 033$$

$$Occurrence probability = exp ( y ) / [ 1 + exp ( y ) ] ,$$

$$where$$

$$y = a 0 + a 1 * [ BB - ( α1 * AA + α2 * CC + α3 * DD ) ] + a 2 * [ EE - ( α 1 * AA + α 2 * CC + α3 * DD ) ] + a 3 * [ FF - ( α1 * AA + α2 * CC + α3 * DD ) ] + a 4 * [ GG - ( α1 * AA + α2 * CC + α3 * DD ) ] + a 5 * [ HH - ( α1 * AA + α2 * CC + α3 * DD ) ]$$

The coefficients and the corresponding ranges are list in the following Coefficient Table 1.

Coefficient Table 1

| Coefficient | Range |
|---|---|
| a0 | −8.2, −2.68 |
| a1 | −0.46, 0.33 |
| a2 | −2.26, −0.61 |
| a3 | 1.23, 2.57 |
| a5 | 0.26, 1.56 |
| α1 | −1.57, −0.26 |
| α2 | −1.15, 0.14 |
| α3 | 1.04, 2.61 |

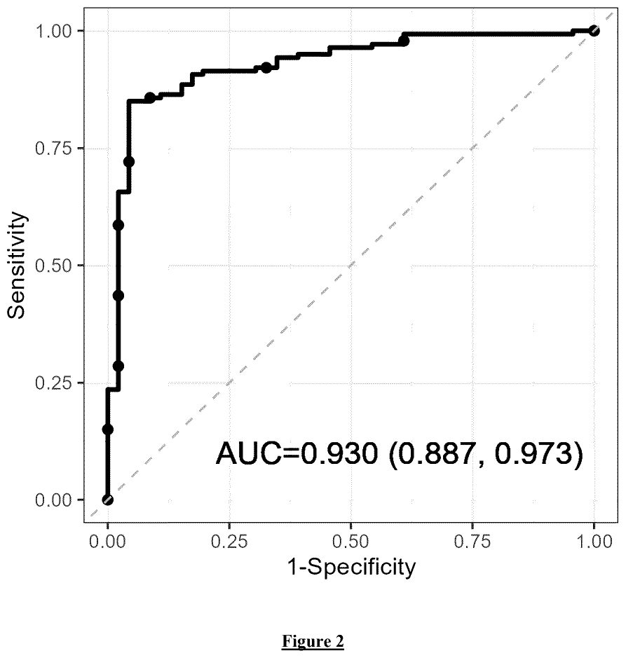

For example, the following is an example calculation of Formula 14 in Table 2. FIG. 2 illustrates the ROC curve of Formula 14 in an embodiment.

$$Formula 014 ( Reference : CC and DD ) . \qquad Formula 014$$

$$Occurrence probability = exp ( y ) / [ 1 + exp ( y ) ] ,$$

$$where$$

$$y = a 0 + a 1 * [ BB - ( α1 * AA + α2 * CC + α3 * DD ) ] + a 2 * [ EE - ( α 1 * AA + α 2 * CC + α3 * DD ) ] + a 3 * [ FF - ( α1 * AA + α2 * CC + α3 * DD ) ] + a 4 * [ GG - ( α1 * AA + α2 * CC + α3 * DD ) ] + a 5 * [ HH - ( α1 * AA + α2 * CC + α3 * DD ) ]$$

The coefficients and the corresponding ranges are list in the following Coefficient Table 2.

Coefficient Table 2

| Coefficient | Range |
|---|---|
| a0 | 2.66, 7.04 |
| a1 | −0.44, 0.43 |
| a2 | −1.47, 0.04 |
| a3 | 2.14, 4.15 |
| a4 | −1.58, −0.57 |
| a5 | 0.26, 1.53 |
| α2 | −0.48, 0.38 |
| α3 | 0.59, 1.6 |

TABLE 2
Performance of formulas ranked by AUC
values in detection of GI cancers

| | | Classifier |
|---|---|---|
| Formula_ID | AUC | Performance |
| Formula_014 | 0.93 | 92.1% |
| Formula_015 | 0.93 | 92.9% |
| Formula_016 | 0.929 | 92.9% |
| Formula_017 | 0.928 | 92.9% |
| Formula_018 | 0.927 | 92.9% |
| Formula_019 | 0.927 | 92.9% |
| Formula_020 | 0.927 | 92.9% |
| Formula_021 | 0.926 | 92.9% |
| Formula_022 | 0.926 | 92.9% |
| Formula_023 | 0.925 | 92.9% |
| Formula_024 | 0.925 | 92.9% |
| Formula_025 | 0.918 | 90.7% |
| Formula_026 | 0.917 | 91.4% |
| Formula_027 | 0.917 | 91.4% |
| Formula_028 | 0.916 | 90.7% |
| Formula_029 | 0.915 | 91.4% |
| Formula_030 | 0.914 | 92.1% |
| Formula_031 | 0.914 | 92.1% |
| Formula_032 | 0.914 | 92.9% |
| Formula_033 | 0.914 | 94.3% |
| Formula_034 | 0.913 | 92.1% |
| Formula_035 | 0.908 | 91.4% |
| Formula_036 | 0.908 | 92.1% |
| Formula_037 | 0.907 | 91.4% |
| Formula_038 | 0.903 | 90.7% |
| Formula_039 | 0.901 | 90.7% |
| Formula_040 | 0.9 | 90.7% |

TABLE 3
Range of coefficients in the Formulae_014-040

| | Range of Coefficients | | | | | | | | |
|---|---|---|---|---|---|---|---|---|---|
| Formulas | [symbol] 0 | [symbol] 1 | [symbol] 2 | [symbol] 3 | [symbol] 4 | [symbol] 5 | [symbol] 1 | [symbol] 2 | [symbol] 3 |
| Formula_{—} | (2.66, 7.04) | (−0.44. 0.43) | (−1.47, 0.04) | (2.14, 4.15) | (−1.58, −0.57) | (0.26, 1.53) | | (−0.48, 0.30) | (0.59, 1.6) |
| 014 | | | | | | | | | |
| Formula_{—} | (−0.41, 2.03) | (0.03, 0.87) | | (1.73, 3.24) | (−1.53, −0.6) | | (−0.91, −0.13) | | (0.8, 1.79) |
| 015 | | | | | | | | | |
| Formula_{—} | (0.39, 2.64) | (0.01, 0.03) | | (1.67, 3.12) | (−1.52, −0.59) | | (−1.01, −0.15) | (−0.62, 0.41) | (0.04, 2.02) |
| 016 | | | | | | | | | |
| Formula_{—} | (4.09, 8.49) | | (−0.9, 0.2) | (3.47, 2.89) | (−1.35, −0.43) | | (−1.83, −0.24) | (−1.41, 0.43) | (1.22, 3.16) |
| 017 | | | | | | | | | |
| Formula_{—} | (1.96, 3.85) | | | (1.59, 2.99) | (−1.32, −0.51) | | (−1.09, −0.09) | | (0.89, 1.85) |
| 018 | | | | | | | | | |
| Formula_{—} | (2.91, 6.5) | | (−0.07, 0.21) | (1.61, 3.12) | (−1.35, −0.43) | | (−1.36, −0.13) | | (0.94, 2.15) |
| 019 | | | | | | | | | |
| Formula_{—} | (3.07, 5.54) | | | (1.45, 2.74) | (−1.32, −0.51) | | (−1.39, −0.16) | (−1.1, 0.36) | (1.07, 2.57) |
| 020 | | | | | | | | | |
| Formula_{—} | (1.31, 3.06) | | | (2, 3.78) | (−1.58, −0.6) | (0.25, 1.16) | | (−0.39, 0.29) | (0.6, 1.46) |
| 021 | | | | | | | | | |
| Formula_{—} | (1.06, 3.31) | (−0.42, 0.43) | | (1.97, 3.82) | (−1.58. −0.6) | (0.7, 1.2) | | (−0.43, 0.32) | (0.59, 1.46) |
| 022 | | | | | | | | | |
| Formula_{—} | (0.70, 2.45) | | | (2.02, 3.81) | (−1.58, −0.61) | (0.25, 1.16) | | | (0.64, 1.31) |
| 023 | | | | | | | | | |
| Formula_{—} | (0.55, 2.07) | (−0.4, 0.46) | | (2, 3.87) | (−1.59, −0.61) | (0.18, 1.10) | | | (0.6, 1.35) |
| 024 | | | | | | | | | |
| Foronda_{—} | (5.68, 11.23) | (−0.06, 0.7) | (−0.91, 0.25) | (2.03, 3.95) | (−1.4, −0.47) | | | | (0.68, 1.57) |
| 025 | | | | | | | | | |
| Formula_{—} | (4.85, 8.33) | (−0.07, 0.67) | | (1.98, 3.77) | (−1.39, −0.513 | | | | (0.66, 1.48) |
| 026 | | | | | | | | | |
| Formula_{—} | (4.47, 0.1) | (−0.05, 0.69) | | (2, 3.82) | (−1.39, −0.52) | | | (−0.34, 0.44) | (0.57, 1.48) |
| 027 | | | | | | | | | |
| Formula_{—} | (5.23, 10.96) | (−0.04, 0.73) | (−0.92. 0.23) | (2.07, 4) | (−1.4, −0.47) | | | (−0.36, 0.5) | (0.56, 1.56) |
| 028 | | | | | | | | | |
| Formula_{—} | (6.24, 12.73) | | (−0.74, 0.31) | (1.83, 3.47) | (−1.24, −0.39) | | | (−0.58, 0.44) | (0.64, 1.8) |
| 029 | | | | | | | | | |
| Formula_{—} | (5.86, 12.03) | | (−0.74, 0.31} | (1.85, 3.5) | (−1.24. −0.39) | | | | (0.73, 1.57) |
| 030 | | | | | | | | | |
| Formula_{—} | (5.66, 10.61) | | | (1.81, 3.38) | (−1.22, −0.45) | | | (−0.54, 0.41) | (0.65,1.65) |
| 031 | | | | | | | | | |
| Formula_{—} | (−8.04, −3.13) | | (−2.27, −0.62) | (1.25, 2.59) | | (9.26, 1.49) | (−1.53, −0.27) | (−1.04, 0.12) | (1.02, 2.53) |
| 037 | | | | | | | | | |
| Formula_{—} | (−8.2, −2.65) | (−0.46,0.33) | (−2.26, −0.62) | (1.23, 2.57) | | (0.26, 1.56) | (−1.57, −0.26) | (−1.15, 0.14) | (1.64, 2.61) |
| 033 | | | | | | | | | |
| Formula_{—} | (5.48, 9.9) | | | (1.83, 3.42) | (−1.21, −0.45) | | | | (0.74, 1.47) |
| 034 | | | | | | | | | |
| Formula_{—} | (−9.01, −3.7) | | (−2.21, −0.6) | (1.42, 2.88) | | (0.21. 1.38) | (−1.13, −0.14) | | (0.75, 1.76) |
| 035 | | | | | | | | | |
| Formula_{—} | (−9.55, −3.35) | (−0.33, 0.5) | (−2.22, −0.6) | (1.43, 2.88) | | (0.13, 1.36) | (−1.14, −0.14) | | (0.72, 1.81) |
| 036 | | | | | | | | | |
| Formula_{—} | (−1.89, 1.02) | (−0.51, 0.22) | (−1.7, −0.21) | (1.66, 3.11) | | (0.1, 1.25) | | (−0.61, 0.34) | (0.6, 1.64) |
| 037 | | | | | | | | | |
| Formula_{—} | (−1.95, 0.76) | | (−1.71, −0.21) | (1.64, 3.19) | | (0.06, 1.1) | | (−0.47, 0.24) | (0.28, 1.55) |
| 028 | | | | | | | | | |
| Formula_{—} | (−2.96, 0.24) | (−0.44, 0.33) | (−1.75, −0.24) | (1.63, 3.22) | | (0.04, 1.17) | | | (0.55, 1.38) |
| 039 | | | | | | | | | |
| Formula_{—} | (−2.89, 0.12) | | (−1.74, −0.24) | (1.67, 3.23) | | (0.06, 1.09) | | | (0.61, 1.34) |
| 040 | | | | | | | | | |

[symbol]

indicates data missing or illegible when filed

In sum, the results indicated that the intended method including the Formula 1-70 is reliable for detection of GI cancers, particularly the gastric cancer, colon cancer, pancreatic cancer and hepatocellular carcinoma, in the blood specimen. The present method can be applied for assisting diagnosis, prognosis, and personalized therapy in the clinic.

### Example 2: Comparison Between a Single Endogenous Reference Marker and A Plurality of Reference Markers Including Endogenous Markers and an Exogenous Marker

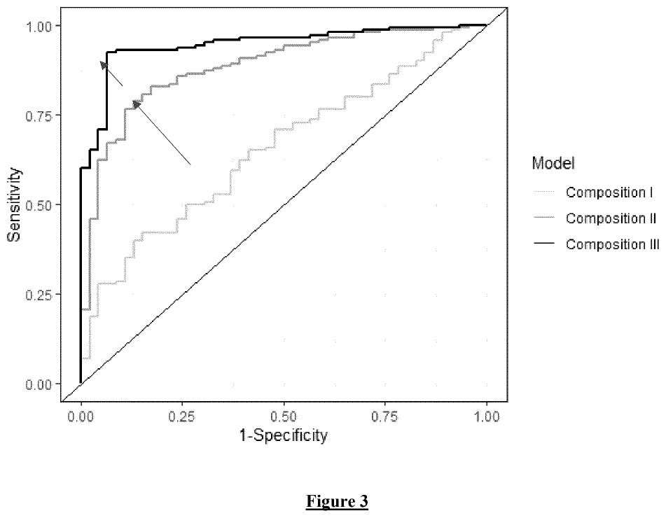

Table 4 indicates three AUC values corresponding three different selections of biomarkers and reference markers. FIG. 3 illustrates comparative ROCs of the three different marker selections.

TABLE 4
Comparative examples:

| Composition | Biomarkers | References | AUC |
|---|---|---|---|
| I | BB | CC | 0.654 |
| II | BB, EE, FF, GG, HH | CC | 0.883 |
| III | BB, EE, FF, GG, HH | AA, CC, DD | 0.945 |

Referring to Table 4 and FIG. 3, Composition I, composed of a single biomarker (BB) and a single reference (CC), showed poor discrimination in the detection of GI cancers, reflected by a low AUC value (0.654). In contrast, Composition II, which applied multiple biomarkers (BB, EE, FF, GG and HH), showed an improved AUC value (0.883) compared to Composition I. Moreover, Composition III, which used multiple references (AA, CC and DD), showed a further increase in AUC value (0.945) as compared with composition II. This result provides a comparative example to show the improvement in the effectiveness of the invented detection panel from the use of single to multiple biomarkers or references.

1. A method of preparing cancer-associated polynucleotide threshold cycle (Ct) value levels useful for assessing a probability of an occurrence of a gastrointestinal (GI) cancer in a patient, comprising:
adding an exogenous miRNA for qPCR normalization to a solution comprising a plurality of different cancer-associated exosomal miRNAs from the patient;
adding a plurality of agents to the solution, to allow the plurality of agents to interact with the plurality of different cancer-associated miRNAs and a plurality of different reference miRNAs including the exogenous miRNA;
generating a plurality of different cancer-associated polynucleotide molecules and a plurality of reference polynucleotide molecules through a quantitative polymerase chain reaction (qPCR), obtaining respective cancer-associated polynucleotide Ct value levels resulted from the qPCR based on the plurality of different cancer-associated polynucleotide molecules and respective reference polynucleotide Ct value levels resulted from the qPCR based on the plurality of different reference polynucleotide molecules, wherein the respective reference polynucleotide Ct value levels comprises a Ct value level resulted from the qPCR based on the exogenous miRNA;
preparing the cancer-associated polynucleotide Ct value levels adjusted with the reference polynucleotide Ct value levels according to Equation (1-2), to assess the probability of the occurrence of the cancer in the patient according to Equation (3) based on the adjusted cancer-associated polynucleotide Ct value levels and assigned coefficients,
wherein the probability of the occurrence of the cancer is assessed according to Equation (3):
$$p = exp ( y ) / ( 1 + exp ( y ) ) \qquad Equation ( 3 )$$
wherein p is the probability of the occurrence of the cancer,
wherein y is calculated from Equation (1-2):
$$y = a 0 + a 1 * [ BB - ( α1 * AA + α2 * CC + α3 * DD ) ] + a 2 * [ EE - ( α 1 * AA + α 2 * CC + α3 * DD ) ] + a 3 * [ FF - ( α1 * AA + α2 * CC + α3 * DD ) ] + a 4 * [ GG - ( α1 * AA + α2 * CC + α3 * DD ) ] + a 5 * [ HH - ( α1 * AA + α2 * CC + α3 * DD ) ] \qquad Equation ( 1 - 2 )$$
wherein
BB, EE, FF, GG, and HH are the obtained cancer-associated polynucleotide Ct value levels, wherein BB corresponds to a level of hsa-miR-191-5p, EE corresponds to a level of hsa-miR-21-5p, FF corresponds to a level of hsa-miR-19a-3p, GG corresponds to a level of hsa-miR-145-5p, and HH corresponds to a level of hsa-miR-23a-3p,
a0, a1, a2, a3, a4, and a5 are the assigned coefficients,
AA, CC, and DD are the obtained reference polynucleotide Ct value levels, wherein AA corresponds to a level of a cel-miR-39-3p being the exogenous miRNA, CC corresponds to a level of a hsa-miR-16-5p, and DD corresponds to a level of a hsa-miR-484, and α1, α2, and α3 are the reference coefficients,
wherein
a0 value is from about −0.41 to about 2.03, a1 value is from about 0.03 to about 0.87, a2 value is 0, a3 value is from about 1.73 to about 3.24, a4 value is from about −1.53 to about −0.6, a5 value is 0, α1 value is from about −0.91 to about −0.13, α2 value is 0, and α3 value is from about 0.8 to about 1.79, or
a0 value is from about 0.39 to about 2.64, a1 value is from about 0.01 to about 0.83, a2 value is 0, a3 value is from about 1.67 to about 3.12, a4 value is from about −1.52 to about −0.59, a5 value is 0, α1 value is from about −1.01 to about −0.15, α2 value is from about −0.62 to about 0.41, and α3 value is from about 0.84 to about 2.02, or
a0 value is from about 4.09 to about 8.49, a1 value is 0, a2 value is from about −0.9 to about 0.2, a3 value is from about 1.47 to about 2.89, a4 value is from about −1.35 to about −0.43, a5 value is 0, α1 value is from about −1.83 to about −0.24, α2 value is from about −1.41 to about 0.43, and α3 value is from about 1.22 to about 3.16, or
a0 value is from about 1.96 to about 3.85, a1 value is 0, a2 value is 0, a3 value is from about 1.59 to about 2.99, a4 value is from about −1.32 to about −0.51, a5 value is about 0, α1 value is from about −1.09 to about −0.09, α2 value is 0, and α3 value is from about 0.89 to about 1.85, or
a0 value is from about 2.91 to about 6.5, a1 value is 0, a2 value is from about −0.87 to about 0.21, a3 value is from about 1.61 to about 3.12, a4 value is from about −1.35 to about −0.43, a5 value is 0, α1 value is from about −1.36 to about −0.13, α2 value is 0, and α3 value is from about 0.94 to about 2.15, or
a0 value is from about 3.07 to about 5.54, a1 value is 0, a2 value is about 0, a3 value is from about 1.45 to about 2.74, a4 value is from about −1.32 to about −0.51, a5 value is 0, α1 value is from about −1.39 to about −0.16, α2 value is from about −1.1 to about 0.36, and α3 value is from about 1.07 to about 2.57, or
a0 value is from about −8.04 to about −3.13, a1 value is 0, a2 value is from about −2.27 to about −0.62, a3 value is from about 1.25 to about 2.59, a4 value is 0, a5 value is from about 0.26 to about 1.49, α1 value is from about −1.53 to about −0.27, α2 value is from about −1.04 to about 0.12, and α3 value is from about 1.02 to about 2.53, or
a0 value is from about −8.2 to about −2.68, a1 value is from about −0.46 to about 0.33, a2 value is from about −2.26 to about −0.61, a3 value is from about 1.23 to about 2.57, a4 value is 0, a5 value is from about 0.26 to about 1.56, α1 value is from about −1.57 to about −0.26, α2 value is from about −1.15 to about 0.14, and α3 value is from about 1.04 to about 2.61, or
a0 value is from about −9.01 to about −3.7, a1 value is 0, a2 value is from about −2.21 to about −0.6, a3 value is from about 1.42 to about 2.88, a4 value is 0, a5 value is from about 0.21 to about 1.38, α1 value is from about −1.13 to about −0.14, α2 value is 0, and α3 value is from about 0.75 to about 1.76, or
a0 value is from about −9.55 to about −3.35, a1 value is from about −0.33 to about 0.5, a2 value is from about −2.22 to about −0.6, a3 value is from about 1.43 to about 2.88, a4 value is 0, a5 value is from about 0.13 to about 1.36, α1 value is from about −1.14 to about −0.14, α2 value is 0, and α3 value is from about 0.72 to about 1.81,
wherein the assessed probability based on the assigned coefficients exhibits at least 0.9 of an area under a receiver operating characteristic (ROC) curve (AUC), wherein a threshold of the occurrence probability is at least about 50%.

2. The method of claim 1, wherein the cancer-associated polynucleotide Ct value levels indicate miRNA levels of the plurality of different cancer-associated miRNAs and respective reference polynucleotide Ct value levels indicate reference miRNA levels of the plurality of different reference miRNAs.

3. (canceled)

4. The method of claim 1, wherein the plurality of different cancer-associated polynucleotide molecules includes a plurality of different copy DNAs (cDNAs) amplified from a plurality of different cancer-associated miRNAs present in a bodily fluid sample.

5-14. (canceled)

15. A method of preparing at least one miRNA threshold cycle (Ct) value level useful for assessing a probability of an occurrence of a gastrointestinal (GI) cancer in a patient comprising:
adding at least one reference miRNA comprising at least one exogenous reference miRNA for qPCR normalization to a bodily fluid sample including a plurality of different cancer-associated miRNAs present in the bodily fluid sample;
processing the bodily fluid sample through a quantitative polymerase chain reaction (qPCR), to determine miRNA Ct value levels of the plurality of different cancer-associated miRNAs and at least one reference miRNA Ct value level of the at least one reference miRNA including the at least one exogenous reference miRNA;
preparing the miRNA Ct value levels adjusted with the at least one reference miRNA Ct value level according to Equation (1-2), to assess the probability of the occurrence of the cancer according to Equation (3), based on the miRNA Ct value levels and assigned coefficients,
wherein the probability of the occurrence of the cancer is assessed based on Equation (3):
$$p = exp ( y ) / ( 1 + exp ( y ) ) \qquad Equation ( 3 )$$
wherein p is the probability of the occurrence of the cancer,
wherein y is calculated from Equation (1-2):
$$y = a 0 + a 1 * [ BB - ( α1 * AA + α2 * CC + α3 * DD ) ] + a 2 * [ EE - ( α 1 * AA + α 2 * CC + α3 * DD ) ] + a 3 * [ FF - ( α1 * AA + α2 * CC + α3 * DD ) ] + a 4 * [ GG - ( α1 * AA + α2 * CC + α3 * DD ) ] + a 5 * [ HH - ( α1 * AA + α2 * CC + α3 * DD ) ] \qquad Equation ( 1 - 2 )$$
wherein
BB, EE, FF, GG, and HH are the obtained cancer-associated polynucleotide Ct value levels, wherein BB corresponds to a level of hsa-miR-191-5p, EE corresponds to a level of hsa-miR-21-5p, FF corresponds to a level of hsa-miR-19a-3p, GG corresponds to a level of hsa-miR-145-5p, and HH corresponds to a level of hsa-miR-23a-3p,
a0, a1, a2, a3, a4, and a5 are the assigned coefficients,
AA, CC, and DD are the obtained reference polynucleotide Ct value levels, wherein AA corresponds to a level of a cel-miR-39-3p being the exogenous miRNA, CC corresponds to a level of a hsa-miR-16-5p, and DD corresponds to a level of a hsa-miR-484, and α1, α2, and α3 are the reference coefficients,
wherein
a0 value is from about −0.41 to about 2.03, a1 value is from about 0.03 to about 0.87, a2 value is 0, a3 value is from about 1.73 to about 3.24, a4 value is from about −1.53 to about −0.6, a5 value is 0, α1 value is from about −0.91 to about −0.13, α2 value is 0, and α3 value is from about 0.8 to about 1.79, or
a0 value is from about 0.39 to about 2.64, a1 value is from about 0.01 to about 0.83, a2 value is 0, a3 value is from about 1.67 to about 3.12, a4 value is from about −1.52 to about −0.59, a5 value is 0, α1 value is from about −1.01 to about −0.15, α2 value is from about −0.62 to about 0.41, and α3 value is from about 0.84 to about 2.02, or
a0 value is from about 4.09 to about 8.49, a1 value is 0, a2 value is from about −0.9 to about 0.2, a3 value is from about 1.47 to about 2.89, a4 value is from about −1.35 to about −0.43, a5 value is 0, α1 value is from about −1.83 to about −0.24, α2 value is from about −1.41 to about 0.43, and α3 value is from about 1.22 to about 3.16, or
a0 value is from about 1.96 to about 3.85, a1 value is 0, a2 value is 0, a3 value is from about 1.59 to about 2.99, a4 value is from about −1.32 to about −0.51, a5 value is about 0, α1 value is from about −1.09 to about −0.09, α2 value is 0, and α3 value is from about 0.89 to about 1.85, or
a0 value is from about 2.91 to about 6.5, a1 value is 0, a2 value is from about −0.87 to about 0.21, a3 value is from about 1.61 to about 3.12, a4 value is from about −1.35 to about −0.43, a5 value is 0, α1 value is from about −1.36 to about −0.13, α2 value is 0, and α3 value is from about 0.94 to about 2.15, or
a0 value is from about 3.07 to about 5.54, a1 value is 0, a2 value is about 0, a3 value is from about 1.45 to about 2.74, a4 value is from about −1.32 to about −0.51, a5 value is 0, α1 value is from about −1.39 to about −0.16, α2 value is from about −1.1 to about 0.36, and α3 value is from about 1.07 to about 2.57, or
a0 value is from about −8.04 to about −3.13, a1 value is 0, a2 value is from about −2.27 to about −0.62, a3 value is from about 1.25 to about 2.59, a4 value is 0, a5 value is from about 0.26 to about 1.49, α1 value is from about −1.53 to about −0.27, α2 value is from about −1.04 to about 0.12, and α3 value is from about 1.02 to about 2.53, or
a0 value is from about −8.2 to about −2.68, a1 value is from about −0.46 to about 0.33, a2 value is from about −2.26 to about −0.61, a3 value is from about 1.23 to about 2.57, a4 value is 0, a5 value is from about 0.26 to about 1.56, α1 value is from about −1.57 to about −0.26, α2 value is from about −1.15 to about 0.14, and α3 value is from about 1.04 to about 2.61, or
a0 value is from about −9.01 to about −3.7, a1 value is 0, a2 value is from about −2.21 to about −0.6, a3 value is from about 1.42 to about 2.88, a4 value is 0, a5 value is from about 0.21 to about 1.38, α1 value is from about −1.13 to about −0.14, α2 value is 0, and α3 value is from about 0.75 to about 1.76, or
a0 value is from about −9.55 to about −3.35, a1 value is from about −0.33 to about 0.5, a2 value is from about −2.22 to about −0.6, a3 value is from about 1.43 to about 2.88, a4 value is 0, a5 value is from about 0.13 to about 1.36, α1 value is from about −1.14 to about −0.14, α2 value is 0, and α3 value is from about 0.72 to about 1.81,
wherein the assessed probability based on the assigned coefficients exhibits at least 0.9 of an area under a receiver operating characteristic (ROC) curve (AUC), wherein a threshold of the occurrence probability is at least about 50%.

16-28. (canceled)
